(19) United States
(12) Patent Application Publication
(10) Pub. No.: US 2016/0264646 A1
(43) Pub. Date: Sep. 15, 2016

(54) PRODUCTION OF HUMAN PULMONARY SURFACTANT PROTEIN B IN PLANTS

(71) Applicant: AZARGEN BIOTECHNOLOGIES (PTY) LTD, Stellenbosch, Western Cape (ZA)

(72) Inventors: Mauritz Venter, Western Cape (ZA); Jacobus Petrus Zwiegelaar, Kuilsrivier, Western Cape (ZA)

(73) Assignee: AZARGEN BIOTECHNOLOGIES (PTY) LTD, Stellenbosch (ZA)

(21) Appl. No.: 14/406,933

(22) PCT Filed: May 16, 2014
(86) PCT No.: PCT/IB2014/061494

§ 371 Date: Dec. 10, 2014

(30) Foreign Application Priority Data
Jun. 24, 2013 (ZA) 2013/04666

(51) Int. Cl. C07K 14/785; C12P 21/02; C12N 15/82; C07K 14/415

(52) U.S. Cl. CPC C07K 14/785; C07K 14/415; C12P 21/02; C12N 15/8257; C07K 2319/02

(57) ABSTRACT

The invention is directed to a method of producing pulmonary surfactant protein-B (SP-B) isoforms fragments and analogues thereof in a plant cell, and to expression cassettes for expression of the SP-B proteins in plants. The expression cassettes may include various elements for improved expression, stability of the expressed protein or efficient purification of the expressed protein, including signal sequences, protease cleavage sites for release of the target protein, trafficking peptides for trafficking of the expressed protein to various plant compartments, and/or various tags.

### BACKGROUND OF THE INVENTION

This invention relates to a method for expression of a human pulmonary surfactant protein B (SP-B) in plants and to SP-B isoforms produced by the method. In particular, the SP-B isoforms are a prepro-SP-B protein and a mature SP-B peptide, or a fragment or analog thereof.

Pulmonary surfactants are detergent-like compounds produced naturally in the lungs and consist of a macromolecular complex of lipids and proteins lining the epithelial surface of the lung. Pulmonary surfactants are produced by type II alveolar cells and are essential to i) lower surface tension and increase lung compliance during breathing, ii) interact with host microbial pathogens and iii) stimulate immune cells in the lungs [Creuwels et al 1997; Goerke 1998]. Pulmonary surfactants are highly conserved among species [Cockshutt and Possmayer 1992; Jobe 1993] and comprise of approximately 90% lipids and 10% proteins [Creuwels et al 1997; Wilson and Notter 2011; Blanco et al 2012]. The genes encoding pulmonary surfactant proteins SP-A (most abundant protein), SP-B, SP-C and SP-D have been studied and deficiency of these proteins are primarily associated with a wide range of respiratory diseases [Mallory 2001; Haataja and Hallman 2002; Whitsett and Weaver 2002; Yurdakök 2004; Clark H, Clark L S (2005].

The surfactant proteins, SP-B and SP-C, are low molecular weight and extraordinarily lipophilic (compared to the hydrophilic, high molecular weight oligomers SP-A and SP-D) and are the most important proteins, strongly associated with surfactant lipids, to affect surface tension properties influencing pulmonary surfactant function [Weaver and Conkright 2001; Parra et al 2011]. Although initially it was believed that all four surfactant proteins were important in facilitating the adsorption of phospholipids to the air-liquid interface of the alveoli to reduce surface tension, it is now known that only SP-B is essential for this function. In particular, deficiency of SP-B is directly associated with respiratory distress syndrome (RDS) that is one of the most important causes of morbidity and mortality in premature-born infants worldwide [Creuwels et al 1997; Engle et al 2008]. RDS can be treated by surfactant replacement therapy (SRT) and SP-B has been identified as the most valuable protein of the two lipophilic proteins, SP-B and SP-C for SRT applications [Wilson and Notter 2011]. Different animal (bovine/pig) and/or synthetic preparations are currently being used for SRT in clinical trials worldwide.

Newer versions of surfactants such as SURFAXIN® are synthetic preparations containing peptides mimicking the natural SP-B protein. SURFAXIN® (lucinactant) is the 5th FDA approved drug to treat RDS in the USA. The other four FDA approved surfactants are EXOSURF® (colfosceril palmitate), SURVANTA® (beractant), CUROSURF® (poractant alpha) and INFASURF® (calfactant) [www.fda.gov]. EXOSURF® is a synthetic surfactant, but is no longer marketed and the other 3 surfactants are animal-derived. Although other delivery methods are being investigated, exogenous surfactant treatments are primarily administered by injection through a catheter directly in the lungs [Halliday 2008; Guttentag and Foster 2011].

In addition to treating RDS, clinical trials using exogenous surfactant have been conducted to treat meconium aspiration syndrome (MAS), congenital diaphragmatic hernia (CDH), bronchopulmonary dysplasia (BPD), genetic disorders of the surfactant system and bronchiolitis, brought on by respiratory syncytial virus (RSV) [Guttentag and Foster 2011]. Other respiratory diseases that have also been targeted for treatment using exogenous surfactant include acute (or adult)-RDS, asthma, viral infections, chronic obstructive pulmonary disease (COPD) [Lusuardi et al 1992; Creuwels et al 1997; Griese M 1999; Stevens and Sinkin 2007; Zuo et al 2008] and other neonatal respiratory disorders [Finer 2004].

New developments in surfactant therapy fall in 3 categories: 1) new indications, 2) new delivery methods and 3) new surfactants [Guttentag and Foster 2011]. Potential drawbacks associated with the current surfactant preparations for RDS as well as other respiratory diseases include: 1) low clinical efficacy, 2) limited product supply, 3) health risk of especially animal-derived products and 4) high cost of product [Mingarro et al 2008; Blanco et al 2012, Ma and Ma 2012]. One of the major strategies highlighted to overcome the inhibition of lung surfactant is to optimize the lipid and surfactant protein content in exogenous preparations [Blanco et al 2012].

There have been numerous developments in synthetic surfactant preparations and/or modification of lipid/protein content for comparative and clinical evaluation and several of the new synthetic surfactants contain either shorter recombinant versions of the native protein or synthetic analogues that mimic the functional properties of the important hydrophobic SP-B and SP-C peptides [Lukovic et al 2006; Almlen et al 2010; Seehase et al 2012; Jordan and Donn 2013]. Although these preparations may show promising clinical results compared to the current formulations [Seehase et al 2012; Jordan and Donn 2013], challenges regarding the synthesis of functional hydrophobic surfactant proteins, especially SP-B, still remain [Zuo et al 2008].

Due to the complexity regarding proper expression and activity of extraordinarily lipophilic surfactant proteins, synthetic surfactants comprising SP-B and/or SP-C analogues may be offered as an alternative to animal-derived natural surfactants, but will not necessarily be clinically superior, especially if SRT applications have been extended to other lung diseases such as Acute Respiratory Distress Syndrome (ARDS) [Zuo et al 2008].

This strongly suggests that there is a need for the development of other technologies to produce functional extraordinarily lipophilic surfactant proteins such as SP-B.

### SUMMARY OF THE INVENTION

According to a first aspect of the invention there is provided a method of producing a pulmonary surfactant protein-B (SP-B) pre-proprotein, or SP-B mature peptide, functional fragment, or analog thereof, in a plant cell, the method comprising the steps of:
providing a nucleic acid sequence comprising a polynucleotide sequence encoding a SP-B pre-proprotein, or SP-B mature peptide, functional fragment, or analog thereof;
(ii) introducing the nucleic acid sequence into an expression vector adapted to express a polypeptide in a plant cell;
(iii) introducing the expression vector of step (ii) into a plant cell;
(iv) expressing the SP-B pre-proprotein, or SP-B mature peptide, fragment of analog thereof in the plant cell of step (iii); and
(v) harvesting the SP-B pre-proprotein, or SP-B mature peptide, or fragment or analog thereof from the plant cell.

Preferably, the SP-B pre-proprotein, or SP-B mature peptide, or fragment thereof is derived from a human gene.

The polynucleotide sequence encoding the SP-B pre-proprotein, or SP-B mature peptide, or functional fragment thereof may be:
(a) 100% identical to any one of SEQ I.D. NOs 2 or 4 (FIGS. 2 and 4);
(b) a variant sequence at least 80% identical to any one of SEQ I.D. NOs 2 or 4 (FIGS. 2 and 4); or
(c) a sequence which hybridises under stringent conditions to the reverse complement of any one of SEQ I.D. NOs 2 or 4 (FIGS. 2 and 4,
   wherein the variant sequence or sequence which hybridises under stringent conditions to the reverse complement encodes a polypeptide capable of lowering surface tension of a lipid bilayer membrane on interaction with phospholipids.

Preferably, the number of hydrophobic amino acid residues encoded by the variant sequence or sequence which hybridises under stringent conditions to the reverse complement of any one of SEQ I.D. NOs 2 or 4 is the same as, or greater than, the number of hydrophobic amino acid residues encoded by any one of SEQ I.D. NOs 2 or 4 (FIGS. 2 and 4).

More preferably, the polynucleotide sequence may have at least 85%, 90%, 95%, 97%, 98%, 99%, or 100% sequence identity to (b) above.

The SP-B pre-proprotein, or SP-B mature peptide sequence may have 100% sequence identity to any one of SEQ I.D. NOs 1 or 3 (FIG. 1 or 3), or may be a variant sequence at least 90% identical to any one of SEQ I.D. NOs 1 or 3 (FIG. 1 or 3), wherein the variant sequence is capable of lowering surface tension of a lipid bilayer membrane on interaction with phospholipids.

Preferably, the number of hydrophobic amino acid residues in the variant sequence is the same as, or greater than, the number of hydrophobic amino acid residues in any one of SEQ I.D. NOs 1 or 3 (FIG. 1 or 3).

More preferably, the SP-B pre-proprotein, or SP-B mature peptide sequence may have at least 95%, 97%, 98%, 99%, or 100% sequence identity to the polypeptide sequences set forth in SEQ ID NO: 1 or 3 (FIG. 1 or 3).

The nucleic acid sequence may further comprise any one or more of the following elements:
(i) a polynucleotide sequence encoding a protease cleavage site, such as any one or more of an enterokinase, chymosin or Tobacco Etch Virus (TEV) protease cleavage site;
(ii) a polynucleotide sequence encoding a tag such as polyhistidine, Leptin, late embryogenesis abundant protein (LEA), Lectin, maltose binding protein (MBP) or glutathione S-transferase (GST), or other tags known to those skilled in the art; and
(iii) a polynucleotide sequence encoding a marker protein for detection, such as YPet, GFP, or others known to those skilled in the art.

Preferably, the one or more elements are combined as a fusion cassette together with the polynucleotide sequence encoding the SP-B pre-proprotein, or SP-B mature peptide, functional fragment, or analog thereof.

The nucleic acid sequence may further comprise any one or more of the following elements:
(i) a plant promoter such as chrysanthemum RbcS1, 35S (such as CaMV35S), or others known to those skilled in the art, and a terminator sequence;
(ii) a polynucleotide sequence encoding a signal peptide such as the signal peptide region of equistatin or of Nicotiana tabacum thionin (NtSP); and
(iii) a polynucleotide sequence encoding a trafficking peptide such as an endoplasmic reticulum (ER)-trafficking peptide e.g., SEKDEL, KDEL, HDEL; an oil body-trafficking peptide (e.g. oleosin), a protein storage vacuole-trafficking peptide (e.g., a Vacuolar Sorting Determinant (VSD) from for example, barley lectin, common bean phaseolin, or soybean β-conglycinin α′ subunit); a plastid, including a chloroplast, chromoplast or leucoplast-trafficking peptide (e.g., a peptide capable of interacting with the thylakoid membrane of a plastid such as the chloroplast targeting signal from the small subunit of Rubisco from Solanum); or another trafficking peptide known to those skilled in the art.

It is to be appreciated that polypeptide targeting to the cytoplasm is also encompassed within the scope of the invention, where there is omission of the inclusion of a tag. Furthermore, other targeting methods for plastids are encompassed within the scope of the invention, such as targeting of a transcript of the polypeptide of the invention to the chloroplast with the use of psbA regulatory 5′-UTR and 3′ UTR regions in the transformation constructs.

In particular, the fusion cassette may consist of any one of the following combinations in the order set out:
(i) MBP-YPet-chymosin-SP-B (for example, SEQ ID NO: 5; corresponding polypeptide sequence set out as SEQ ID NO: 6) (FIGS. 5 and 6);
(ii) Lectin-YPet-TEV-SP-B-TEV (for example, SEQ ID NO: 7; corresponding polypeptide sequence set out as SEQ ID NO: 8) (FIGS. 7 and 8);
(iii) YPet-TEV-SP-B-TEV-polyhistidine (for example, SEQ ID NO: 9; corresponding polypeptide sequence set out as SEQ ID NO: 10) (FIGS. 9 and 10);
(iv) YPet-chymosin-SP-B-TEV-Leptin (for example, SEQ ID NO: 11; corresponding polypeptide sequence set out as SEQ ID NO: 12) (FIGS. 11 and 12);
(v) LEA-chymosin-SP-B-TEV-YPet (for example, SEQ ID NO: 13; corresponding polypeptide sequence set out as SEQ ID NO: 14) (FIGS. 13 and 14); and
(vi) YPet-chymosin-SP-B-TEV-LEA (for example, SEQ ID NO: 15; corresponding polypeptide sequence set out as SEQ ID NO: 16) (FIGS. 15 and 16).

The fusion cassette may consist of or a polynucleotide sequence that is at least 80% identical to:
(a) any one of the polynucleotide sequences set out in SEQ ID NOs: 5, 7, 9, 11, 13, or 15; or
(b) a sequence which hybridises under stringent conditions to the reverse complement of any one of the polynucleotide sequences set out in SEQ ID NOs: 5, 7, 9, 11, 13, or 15.

Further in particular, the nucleic acid sequence may comprise, in the order set out: a promoter sequence-signal peptide-any one of the fusion cassette combinations-trafficking peptide-terminator sequence.

For example, the nucleic acid sequence may comprise, in the order set out: RbcS1 promoter sequence-equistatin-any one of the fusion cassette combinations-KDEL-RbcS1 terminator sequence.

Optionally, the polynucleotide sequences may be codon optimised for expression in plant cells.

Optionally, the expression vector of step (ii) may further comprise a polynucleotide sequence encoding a suppressor protein adapted to inhibit post-transcriptional gene silencing in a plant cell. Alternatively, step (iii) may further include introducing into the plant cell a second plant vector comprising a polynucleotide sequence encoding a suppressor protein adapted to inhibit post-transcriptional gene silencing in the plant cell. For example, the suppressor protein may be the NSs protein of the tomato spotted wilt virus or the p19 of tomato bushy stunt virus, or others known to those skilled in the art.

Preferably, in step (iv) the expressed pulmonary surfactant protein-B pre-proprotein is retained in the ER of the plant cell.

Alternatively, in step (iv) the expressed pulmonary surfactant protein-B mature peptide is targeted to a plant component such as a plastid, oil body, protein storage vacuole or the cytoplasm of the plant cell.

The plant expression vector of step (ii) of the method may be an Agrobacterium tumefaciens vector.

Step (iii) of the method may comprise stable transformation of a plant cell by the introduction of the expression vector of step (ii) into the genetic material of the plant cell. For example, the method of introduction may be by agrobacterium transformation or agroinfiltration or other methods known to those skilled in the art.

The plant cell may be a plurality of plant cells in suspension culture, plant cells in tissue culture, plant cells in a leaf of a plant or a transgenic plant or any part thereof.

According to a further aspect of the invention, there is provided a polypeptide fusion cassette comprising a SP-B pre-proprotein or SP-B mature peptide, functional fragment of analog thereof produced by the method of the invention.

According to a further aspect of the invention, there is provided a plant-based expression vector comprising the nucleic acid sequence according to the invention.

According to a further aspect of the invention, there is provided a plant cell comprising a plant-based expression vector or a SP-B pre-proprotein or SP-B mature peptide, functional fragment, or analog thereof produced by the method of the invention.

According to a further aspect of the invention, there is provided a SP-B pre-proprotein or SP-B mature peptide, or fragment thereof according to the invention for use in a method of preventing and/or treating a respiratory disease, condition or disorder in a subject.

According to a further aspect of the invention, there is provided a use of the SP-B pre-proprotein or SP-B mature peptide, functional fragment or analog thereof produced by the method of the invention for use in the manufacture of a medicament for use in a method of preventing and/or treating a respiratory disease, condition or disorder in a subject.

According to a further aspect of the invention, there is provided a method of preventing and/or treating a respiratory disease, condition or disorder in a subject, the method comprising a step of administering a prophylactically or therapeutically effective amount of a SP-B pre-proprotein or SP-B mature peptide, functional fragment or analog thereof produced by the method of the invention to the subject.

Preferably, the SP-B pre-proprotein or SP-B mature peptide, or fragment thereof is a human SP-B pre-proprotein or SP-B mature peptide, or fragment thereof.

The respiratory disease, condition or disorder may include any one or more of the following: respiratory distress syndrome (RDS) in infants, meconium aspiration syndrome (MAS), congenital diaphragmatic hernia (CDH), bronchopulmonary dysplasia (BPD), genetic disorders of the surfactant system and bronchiolitis, for example brought on by respiratory syncytial virus (RSV), acute (or adult)-RDS, asthma, viral infections, chronic obstructive pulmonary disease (COPD) and other neonatal respiratory disorders.

Preferably, the respiratory disease, condition or disorder is respiratory distress syndrome (RDS) in premature born infants.

The subject is preferably a human.

### BRIEF DESCRIPTION OF THE DRAWINGS

FIG. 1 shows the polypeptide sequence (381 aa) of the prepro-SP-B polypeptide (SEQ ID NO: 1);

FIG. 2 shows a cDNA sequence (1146 bp) encoding the prepro-SP-B polypeptide (SEQ ID NO: 2);

FIG. 3 shows the polypeptide sequence (79 aa) of the mature SP-B peptide (SEQ ID NO: 3);

FIG. 4 shows a cDNA sequence 237 bp) encoding the mature SP-B peptide (SEQ ID NO: 4);

FIG. 5 shows a cDNA sequence encoding the fusion cassette consisting of MBP-YPet-chymosin-SP-B (SEQ ID NO: 5);

FIG. 6 shows the polypeptide sequence of the fusion cassette consisting of MBP-YPet-chymosin-SP-B (SEQ ID NO: 6);

FIG. 7 shows a cDNA sequence encoding the fusion cassette consisting of Lectin-YPet-TEV-SP-B-TEV (SEQ ID NO: 7);

FIG. 8 shows the polypeptide sequence of the fusion cassette consisting of Lectin-YPet-TEV-SP-B-TEV (SEQ ID NO: 8);

FIG. 9 shows a cDNA sequence encoding the fusion cassette consisting of YPet-TEV-SP-B-TEV-polyhistidine (SEQ ID NO: 9);

FIG. 10 shows the polypeptide sequence of the fusion cassette consisting of YPet-TEV-SP-B-TEV-polyhistidine (SEQ ID NO: 10);

FIG. 11 shows a cDNA sequence encoding the fusion cassette consisting of YPet-chymosin-SP-B-TEV-Leptin (SEQ ID NO: 11);

FIG. 12 shows the polypeptide sequence of the fusion cassette consisting of YPet-chymosin-SP-B-TEV-Leptin (SEQ ID NO: 12);

FIG. 13 shows a cDNA sequence encoding the fusion cassette consisting of LEA-chymosin-SP-B-TEV-Ypet (SEQ ID NO: 13);

FIG. 14 shows the polypeptide sequence of the fusion cassette consisting of LEA-chymosin-SP-B-TEV-Ypet (SEQ ID NO: 14);

FIG. 15 shows a cDNA sequence encoding the fusion cassette consisting of YPet-chymosin-SP-B-TEV-LEA (SEQ ID NO: 15);

FIG. 16 shows the polypeptide sequence of the fusion cassette consisting of YPet-chymosin-SP-B-TEV-LEA (SEQ ID NO: 16);

FIG. 17 shows a cDNA sequence (93 bp) encoding the tobacco thionin like protein (NtSP) signal peptide (SEQ ID NO: 17);

FIG. 18 shows the polypeptide sequence (31 aa) of the tobacco thionin like protein (NtSP) signal peptide (SEQ ID NO: 18);

FIG. 19 shows a cDNA sequence encoding the equistatin signal peptide (SEQ ID NO: 19);

FIG. 20 shows the polypeptide sequence of the equistatin signal peptide (SEQ ID NO: 20);

FIG. 21 shows the pART27_preproSP-B_SEKDEL expression vector;

FIG. 22 shows the pART27-NtSP-His-mpSP-B expression vector;

FIG. 23 shows an embodiment of the nucleic acid sequence of the invention cloned into the plant expression vector, ImpactVector 1.3;

FIG. 24 shows RT-PCR analysis of (A) lane 1-wild-type plant (non-transgenic) control; lanes 2 to 11-plant lines 2, 7, 12, 18, 20, 22, 26, 38, 40 and 45 of the preproSP-B transcript, and (B) lanes 1 to 14-plant lines 1, 6, 10, 13, 15, 17, 18, 22, 24, 25, 27, 31, 32 and 37 of the mpSP-B transcript. Top panels for both SP-B transcripts show positive control β-actin activity in respective plant lines;

FIG. 25 A) shows a representative western blot of human recombinant preproSB-B protein extracted from plant leaves: left panel-non-transgenic wild-type tobacco control; right panel-transgenic tobacco plant line 45. Protein samples volumes were from 25 μl down to 5 μl; B) shows a representative western blot of human recombinant mpSP-B and preproSP-B plant lines: lane C+-SP-B positive control (Abnova); lane 2-non-transgenic wild-type tobacco control; lane 3-transgenic mpSP-B tobacco plant line 37; lanes 4 and 5-transgenic preproSP-B tobacco plant lines 40 and 45;

FIG. 26 shows a representative western blot of Fc_3 with an expected size of 80.7 kDa extracted from plant leaves of transgenic plant lines. Lane M is protein standard (Life Technologies), lane wt=non-transgenic wild-type tobacco, lane C+=purified YPet fluorescent protein positive control, lanes 1 to 9=transgenic plant lines;

FIG. 27 shows a representative western blot of Fc_7 with an expected size of 67.4 kDa extracted from plant leaves of transgenic plant lines. Lane M is protein standard (Life Technologies), lane wt=non-transgenic wild-type tobacco, lane C+=purified YPet fluorescent protein positive control, lanes 1 to 9=transgenic plant lines;

FIG. 28 shows a representative western blot of Fc_9 with an expected size of 40.6 kDa extracted from plant leaves of transgenic plant lines. Lane M is protein standard (Life Technologies), lane wt=non-transgenic wild-type tobacco, lane C+=purified YPet fluorescent protein positive control, lanes 1 to 9=transgenic plant lines;

FIG. 29 shows a representative western blot of Fc_10 with an expected size of 50 kDa extracted from plant leaves of transgenic plant lines. Lane M is protein standard (Life Technologies), lane wt=non-transgenic wild-type tobacco, lane C+=purified YPet fluorescent protein positive control, lanes 1 to 9=transgenic plant lines;

FIG. 30 shows a representative western blot of Fc_12 with an expected size of 50 kDa extracted from plant leaves of transgenic plant lines. Lane M is protein standard (Life Technologies), lane wt=non-transgenic wild-type tobacco, lane C+=purified YPet fluorescent protein positive control, lanes 1 to 9=transgenic plant lines; and

FIG. 31 shows a representative western blot of Fc_13 with an expected size of 50 kDa extracted from plant leaves of transgenic plant lines. Lane M is protein standard (Life Technologies), lane wt=non-transgenic wild-type tobacco, lane C+=purified YPet fluorescent protein positive control, lanes 1 to 9=transgenic plant lines.

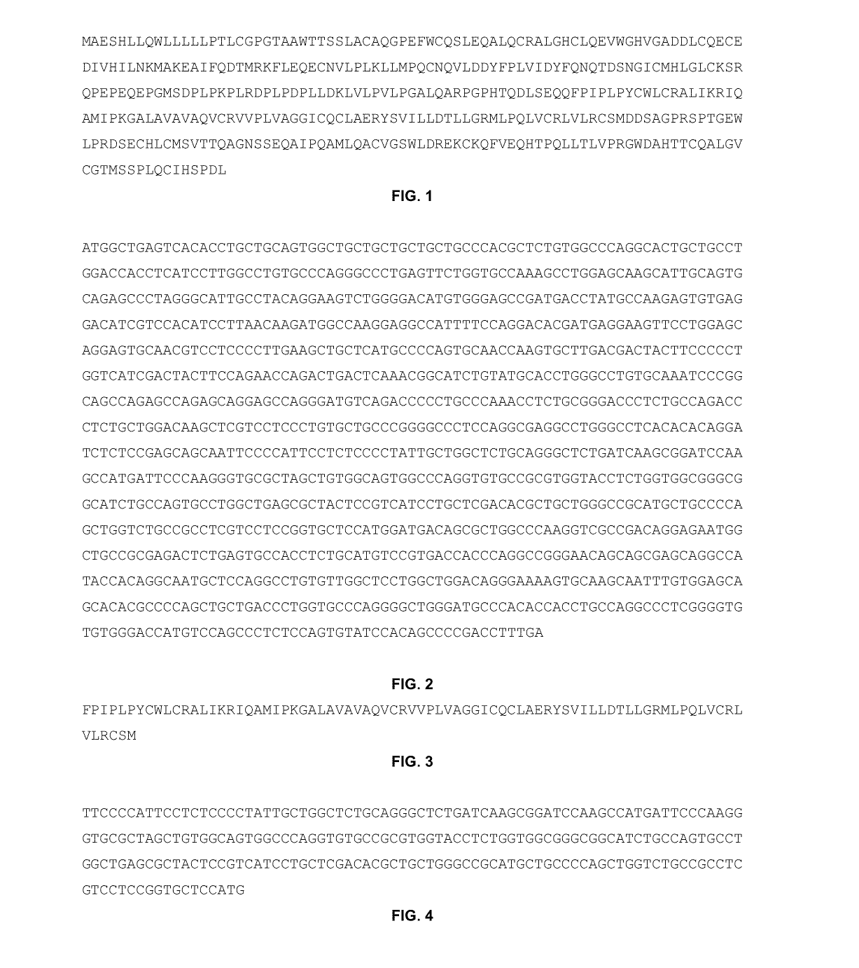

### DETAILED DESCRIPTION OF THE INVENTION

The current invention provides a method for expression of a human pulmonary surfactant protein B (SP-B) in plants and to SP-B isoforms produced by the method and to functional fragments of these isoforms. In particular, the SP-B isoforms are a prepro-SP-B protein and a mature SP-B peptide, or a functional fragment thereof.

Generally, recombinant proteins are currently produced by bacterial, yeast, insect- and/or mammalian cell production platforms [Drugmand et al 2012; Huang et al 2012; Kim et al 2012; Mattanovich et al. 2012] of which bacterial (E. coli) and mammalian (usually Chinese Hamster Ovary_CHO) cells are the predominant systems used for commercial production. Expression of recombinant SP-C versions in E. coli have been reported [Lukovic et al 2006], but traditionally, mammalian cell systems, specifically CHO cells, have been preferred. Over the years these different production platforms have been optimized to achieve higher protein production levels and improved industrial scalability [Berlec and Strukelj 2013]. However, these systems have a variety of important drawbacks that include partially active or non-functional products (especially in E. coli where post-translational modification is lacking), high cost of production and safety issues (i.e. animal pathogens, allergic responses).

Furthermore, a recent study at the University of Michigan was conducted to explore and compare the views of neonatologists and parents of newborns regarding the use of animal-derived medications. The study revealed that the majority (92%) of neonatologists were concerned about exposure of newborns to animal-derived pharmaceutical agents and 58% of the parents chose a non-animal derived version with safety as the prime concern.

Compared to current bacterial, yeast, insect and mammalian recombinant protein production systems, the use of plant cells to express human recombinant proteins may offer greater advantages with regard to 1) safety: no immunogenic responses or animal/bacterial-derived contamination, 2) complexity: plants are higher organisms with protein assembly mechanisms similar to humans, which can synthesize complex biopharmaceutical proteins (unlike bacterial fermentation systems that lack post-translational modification processes), 3) cost and scalability: plant-based systems allow for significantly lower facility and production costs (compared to mammalian cell-based systems) and production can easily be scaled up to accommodate increased demands.

A wide variety of plant-based recombinant biopharmaceutical compounds such as therapeutic proteins and vaccine antigens have been expressed using different plant-based expression hosts, platforms and tissues, but there are many factors, genetic and environmental, that influence successful plant-based recombinant protein production, and each system must be optimised and empirically tested for the specific protein to be produced, particularly for proteins that are highly lipophilic which have unique challenges associated with expression, correct folding and purification.

The three major plant-based production platforms are: 1) stable transformation, integration of foreign DNA in plant genome, 2) transient transformation, using viral vector and 3) plant cell culture. Each platform has a distinct advantage or disadvantage for a particular protein candidate, using a specific plant host, regarding production cost and time, scalability and regulatory compliance.

An important factor when determining whether a plant-based production platform is appropriate for production of a particular biopharmaceutical protein candidate, is the need to be able to express biopharmaceutical proteins at commercially viable levels. Even more importantly, to enter clinical development, plant-based production platforms must conform to good manufacturing practice (GMP) regulations.

SP-B and analogs thereof can be synthetically produced and function has been shown to be dependant on the interaction of the lipophilic protein with phospholipids to lower surface tension. Indeed, several of the new synthetic surfactants contain either shorter recombinant versions of the native protein or synthetic analogues that mimic the functional properties of the important hydrophobic SP-B and SP-C peptides [Lukovic et al 2006; Almlen et al 2010; Seehase et al 2012; Jordan and Donn 2013]. It was found that the early SP-B truncated analogs that were similar in sequence to the C-terminal of native mature SP-B had similar biological function (when combined with phospholipids) to naturally occurring SP-B. It was therefore determined that activity is not primarily involved with SP-B peptide folding or conformation, but rather the presence of the necessary hydrophobic residues in the final sequence.

Compared to animal-derived and/or synthetic compounds comprising synthetic peptide analogues that only mimic the natural human surfactant protein, plant-based recombinant human surfactant proteins may address some of the drawbacks associated with current mammalian produced and synthetic surfactant preparations.

Of the two extraordinarily lipophilic surfactant proteins, SP-B plays a key role in the maturation of surfactant and is more active than SP-C concerning biophysical interaction with lipids and physiological function in lung surfactant. Addition of SP-B enhances phospholipid mixture activity and lowers surface tension to reverse neonatal respiratory failure [Pryhuber 1998].

The applicants thus sought to investigate whether a plant-based platform might potentially offer a means for the production of functional copies of the important exceptionally lipophilic surfactant protein SP-B. The SP-B protein is naturally processed in the lungs from a 381 amino acid, 40-kDa pre-proprotein (SEQ ID NO: 1; corresponding polynucleotide sequence set out as SEQ ID NO: 2) to a 79 amino acid, 8-kDa mature peptide (SEQ ID NO: 3; corresponding polynucleotide sequence set out as SEQ ID NO: 4) [Pryhuber 1998]. The applicant was able to successfully demonstrate the expression of both SP-B protein isoforms in transformed transgenic plants. It is expected that the method would be similarly successful with functional fragments and analogs of the SP-B protein isoforms.

A polynucleotide sequence encoding a polypeptide of interest (e.g., a SP-B pre-proprotein, a SP-B mature peptide, or fusion cassette thereof), may be or contain, a polynucleotide sequence, having at least 80% sequence identity (e.g., at least 85%, 90%, 95%, 97%, 98%, 99%, or 100% sequence identity) to the polynucleotide sequences encoding the corresponding wild-type polypeptides thereof (i.e. those set forth in SEQ ID NOs: 2, 4, 5, 7, 9, 11, 13, 15, 17 or 19), with the proviso that the variant sequence is also capable of lowering surface tension of a lipid bilayer membrane on interaction with phospholipids. In particular, the number of hydrophobic residues in the variant sequence should be the same as, or greater than, the corresponding wild-type SP-B polypeptide or fragment thereof as it occurs naturally. In addition, the functional polypeptides of interest may have at least 90% sequence identity (e.g., at least 95%, 97%, 98%, 99%, or 100% sequence identity) to the naturally occurring polypeptide sequences (i.e. those set forth in SEQ ID NOs: 1, 3, 6, 8, 10, 12, 14, 16, 18 or 20), with the proviso that that the variant sequence is also capable of lowering surface tension of a lipid bilayer membrane on interaction with phospholipids. In particular, the number of hydrophobic residues in the variant sequence should be the same as, or greater than, the corresponding wild-type SP-B polypeptide, or fragment thereof as it occurs naturally. The means for determination of percent identity between a particular polynucleotide or amino acid sequence and the polynucleotide or amino acid sequence set forth for a polynucleotide or protein is well known to those skilled in the art may be performed with multiple commercially available sequence analysis programs such as the BLAST programme and others known to those skilled in the art such as provided on the U.S. government's National Center for Biotechnology Information web site (www.ncbi.nlm.nih.gov).

The polynucleotide sequence identity compared to polynucleotide encoding the SP-B pre-proprotein or SP-B mature peptide may exist over a region of the sequence that is about 100, 150, 200, 250, 300, 350, 400, 450, 500, 550, 600, 650, 700, 750, 800, 850, 900, 950, 1000 or more consecutive nucleotide residues in length. The amino acid identity compared to a SP-B pre-proprotein or SP-B mature peptide may exist over a region of the sequence that is about 50, 100, 150, 200, 250, 300, 350 or more consecutive amino acid residues in length.

It is further to be appreciated that a number of nucleic acids can encode a polypeptide having a particular amino acid sequence and such degeneracy of the genetic code is well known to the art. Such variations in degeneracy are included in the scope of the invention. For example, codons in the coding sequence for a given polypeptide can be modified such that optimal expression in a particular species (e.g., plants) is obtained, using appropriate codon bias tables for that species.

The scope of the invention further covers polynucleotide sequences which are able to hybridise under high stringency to a complement of SEQ I.D. NOs: 2, 4, 5, 7, 9, 11, 13, 15, 17 or 19 with the proviso that the variant sequence encodes a polypeptide that is capable of lowering surface tension of a lipid bilayer membrane on interaction with phospholipids. In particular, the number of hydrophobic residues in the polypeptide encoded by the variant sequence should be the same as, or greater than, the corresponding wild-type SP-B polypeptide or fragment thereof as it occurs naturally. Hybridization conditions are well known to those skilled in the art. Highly stringent conditions are defined as equivalent to hybridization in 6× sodium chloride/sodium citrate (SSC) at 45° C., followed by a wash in 0.2×SSC, 0.1% SDS at 65° C.

It is also to be appreciated that any of the synthetic sequences or analogues of SP-B presently produced, such as those described in Almlen et al., 2010 (CWLCRALIKRIQAMIPKGGRMLPQLVCRLVLRCS, also known as Mini-B; LLLKLLKLLLKLLKLLLKLLL (KL-peptide); LLKLKLLLLLLLKLKLLLLLLLKLKLL (KLK-peptide)), Seehase et al., 2012 (CWLCRALIKRIQALIPKGGRLLPQLVCRLVLRCS, a synthetic SP-B analog), or Jordan and Donn 2013 could similarly be produced in plants using the method of the invention.

As previously indicated, functional activity of an SP-B analog or fragment is not primarily involved with SP-B peptide folding or conformation, but rather the presence of hydrophobic residues in the final sequence. As previously indicated, functional activity of an SP-B analog or fragment is primarily associated with the presence of hydrophobic residues in the final sequence. Functional fragments or analogs of the SP-B polypeptides of the invention may therefore be any SP-B pre-proprotein or mature SP-B peptide fragment or any polypeptide analog thereof comprising hydrophobic amino acid residues, which, when they appropriately interact with specific phospholipids, will lower surface tension of a lipid bilayer membrane.

As unreliable transgene expression may be problematic when using conventional plant-based expression constructs for expression of a highly lipophilic, membrane bound protein, such as SP-B, the applicant investigated whether it might be possible to improve the expression levels and purification efficiencies of SP-B by fusion of the SP-B polypeptide to various heterologous polypeptides including protein tags and trafficking polypeptides in a fusion cassette. Such a fusion tag combination can comprise several types of tags for purification, such as affinity tags, for example, FLAG, polyhistidine, hemagluttanin (HA), glutathione-S-transferase (GST), or maltose-binding protein (MBP); tags for detection (such as yellow fluorescent protein (YPet), luciferase, green fluorescent protein (GFP), or chloramphenicol acetyl transferase (CAT); and tags for enhanced expression and stability (such as an expression protein partner tag, for example, Leptin, late embryogenesis abundant protein (LEA), Lectin, maltose binding protein (MBP) or glutathione S-transferase (GST).

Trafficking polypeptides may in particular include those for ER targeting such as KDEL, SEKDEL, or HDEL (with or without a signal peptide such as equistatin), or others known to those skilled in the art. However, tags for targeting to various other compartments such as oil bodies (e.g. recombinant polypeptide targeting to seeds of transgenic plants by fusion with oleosin), protein storage vacuoles (e.g., by fusion of a recombinant polypeptide with a Vacuolar Sorting Determinant (VSD) from for example, barley lectin, common bean phaseolin, or soybean β-conglycinin α′ subunit), plastids, including chloroplasts, chromoplasts and leucoplasts (e.g. through fusion of a polypeptide with a peptide capable of interacting with the thylakoid membrane of a plastid such as the chloroplast targeting signal from the small subunit of Rubisco from Solanum) are well known to those skilled in the art and may also be used. It is to be appreciated that polypeptide targeting to the cytoplasm is also encompassed within the scope of the invention, where there is omission of the inclusion of a tag. Furthermore, other targeting methods for plastids are encompassed within the scope of the invention, such as targeting of a transcript of the polypeptide of the invention to the chloroplast with the use of psbA regulatory 5′-UTR and 3′ UTR regions in the transformation constructs.

Furthermore, the applicant investigated whether inclusion of a protease cleavage site flanking the SP-B within the fusion cassette for splicing the SP-B from the fusion partners may enhance expression and purification of the SP-B. All fusions were thus separated by linker peptide(s) containing a protease recognition site. The protease recognition site(s) included were selected from tobacco etch virus (TEV) or chymosin or both, although an enterokinase cleavage site may also be used.

Additionally, the applicant included in the plant expression vector a signal peptide, such as the signal peptide region of equistatin or of Nicotiana tabacum thionin (NtSP). The plant promoter used may be the chrysanthemum RbcS1, 35S (such as CaMV35S), or other plant promoters known to those skilled in the art, together with an appropriate terminator sequence such as the RbcS1 terminator, the Nos polyA or the CaMV35S polyA terminator sequence.

In particular, the applicant designed six different polypeptide fusion cassettes and was able to successfully demonstrate transgenic plant-based expression of the mature processed 8 kDa SP-B polypeptide for expression analysis in Nicotiana tobaccum (tobacco) plants. However, it is to be appreciated that the fusion cassettes may also be used for expression of the prepro-SP-B polypeptide.

The invention will be described by way of the following examples which are not to be construed as limiting in any way the scope of the invention.

### EXAMPLES

### Example 1

### 1. Isolation and Cloning of the Human Prepro-SP-B cDNA into a Plant Transformation Vector

The human SP-B cDNA (1146 bp) encoding the prepro-SPB protein (GenBank acc NM_000542) (SEQ ID NO: 1; FIG. 1), was amplified from human lung cDNA (Ambion FirstChoice PCR-Ready) using the primers: preproSP-B_frw (5′-ATG GCT GAG TCA CAC CTG CTG-3′) (SEQ ID NO: 21) and preproSP-B_rev (5′-TCA AAG GTC GGG GCT GTG GAT AC-3′) (SEQ ID NO: 22). PCR amplification of prepro-SPB cDNA included a predenaturation at 94° C. for 30 sec followed by 35 cycles of amplification (94° C. denaturation, 30 sec; 50° C. annealing, 30 sec; 72° C. polymerization, 1 min 30 sec). PCR products were visualized in ethidium bromide-stained 1% (w/v) agarose gels (results not shown), extracted and cloned in a pGEM®-T Easy Vector (Promega Corporation, Madison, USA). Selected clones were sequenced (ABI PRISM® 3100 genetic analyser) using the ready reaction kit with AmpliTaq® DNA polymerase (The Perkin Elmer Corporation, Norwalk, USA).

A second prepro-SPB PCR fragment was generated using the primers: preproSP-B27_frw (5′-CCCCGAATTCATGGCTGAGTCACACCTGCTG-3′) (SEQ ID NO: 23) incorporating a 5′-EcoRI restriction site and preproSP-B27_rev (5′-ggggAAGCTTTCAaagctcgtccttctcgctGTGATGGTGGTGGTGATGTTTGTCATCGT CATCAAGGTCCGGGCTGTGGATACACT-3′) (SEQ ID NO: 24) incorporating an enterokinase site, His-Tag® sequence, a SEKDEL sequence for ER retention, TGA-stop codon and a HindIII restriction site at the 3′-end. PCR amplification using Supertherm Taq (Medox Biotech, India), included a predenaturation at 94° C. for 2 min followed by 35 cycles of amplification (94° C. denaturation, 30 sec; 50° C. annealing, 30 sec; 72° C. polymerization, 2 min). PCR product was visualized in ethidium bromide-stained 0.8% (w/v) agarose gel, extracted and digested with EcoRI and HindIII restriction enzymes. Digested PCR product was cloned into pART7 casette [Gleave 1992] prepared with EcoRI and HindIII followed by sub-cloning into the NotI sites of pART27 to yield pART27_preproSP-B_SEKDEL (see FIG. 21). Selected pART27 clones were sequenced (ABI PRISM® 3100 genetic analyser) using the ready reaction kit with AmpliTaq® DNA polymerase (The Perkin Elmer Corporation, Norwalk, USA) to confirm the preproSP-B_SEKDEL sequence.

### 2. Isolation and Cloning of the Mature SP-B Peptide (mpSP-B) cDNA into the Plant Transformation Vectors

The cDNA region (237 bp) encoding the mature SP-B peptide (SEQ ID NO: 3; FIG. 3) was amplified from human lung cDNA (Ambion FirstChoice PCR-Ready) using the primers: mpSP-B_frw (5′-GGCTCGAGTTCCCCATTCCTCTCCCCTA-3′) (SEQ ID NO: 25) and mpSP-B_rev (5′-GGCAGATCTCATGGAGCACCGGAGGACGA-3′) (SEQ ID NO: 26) generating XhoI and BglII sites at the 5′- and 3′ ends respectively. PCR amplification using Elongase® enzyme mix, included predenaturation at 94° C. for 2 min followed by 35 cycles of amplification (94° C. denaturation, 30 sec; 60° C. annealing, 30 sec; 68° C. polymerization, 1 min). PCR products were visualized in ethidium bromide-stained 1% (w/v) agarose gels (results not shown), extracted and cloned in a pGEM®-T Easy Vector (Promega Corporation, Madison, USA). Selected clones were sequenced (ABI PRISM® 3100 genetic analyser) using the ready reaction kit with AmpliTaq® DNA polymerase (The Perkin Elmer Corporation, Norwalk, USA).

A pET-14b vector (Novagen) containing an N-terminal His-Tag® sequence followed by a thrombin cleavage site was linearized at the XhoI restriction site. The mpSP-B fragments, digested with XhoI and BglII, were gel purified and resuspended in a ligation mix containing T4 DNA ligase, ligase buffer (Promega Corporation, Madison, USA) and linearized pET-14b vector to yield pET14B-mpSP-B. The ligation mixture was not transformed into competent cells, but rather was used as template for the construction of the downstream plant expression vectors.

A signal peptide region (SEQ ID NO: 18; FIG. 18) of the Nicotiana tabacum thionin (Genbank acc AB034956; cDNA (SEQ ID NO: 17; FIG. 17) was amplified from tobacco cDNA using the primers: NtSP-FRW (5′-GGCTCGAGATGGCAAACTCCATGCGCTTCTTT-3′ (SEQ ID NO: 27) incorporating a 5′-XhoI restriction site) and NtSP-REV (5′-CCAAGCTTTGCCTCTGCAATTGTCATTG-3′ (SEQ ID NO: 28) to incorporate a HindIII restriction site at the 3′-end of the signal peptide). The tobacco thionin signal peptide targets expressed peptides to the apoplastic region in the plant cell. PCR amplification using Expand® enzyme mix, included predenaturation at 95° C. for 5 min followed by 35 cycles of amplification (95° C. denaturation, 30 sec; 55° C. annealing, 30 sec; 72° C. polymerization, 30 sec). PCR products were extracted and cloned in a pGEM®-T Easy Vector (Promega Corporation, Madison, USA) to yield pGEM-NtSP. Positive colonies were identified by restriction digest with XhoI and HindIII. The Nt-sp fragment was cloned into pART7 casette [Gleave 1992] prepared with XhoI and HindIII and sub-cloned into the NotI sites of pART27 to yield to yield pART27_NtSP.

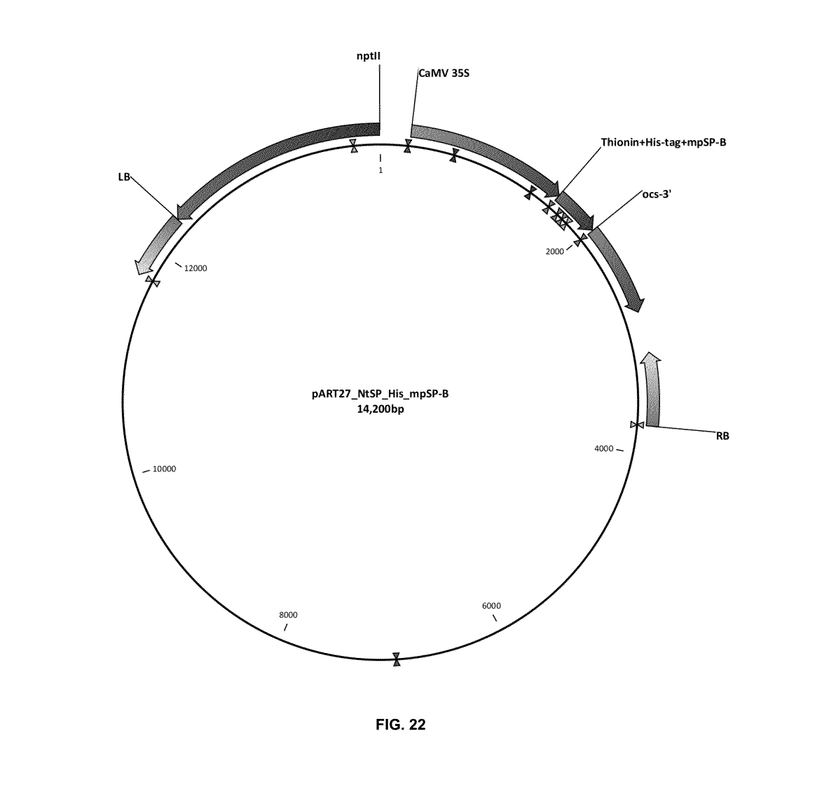

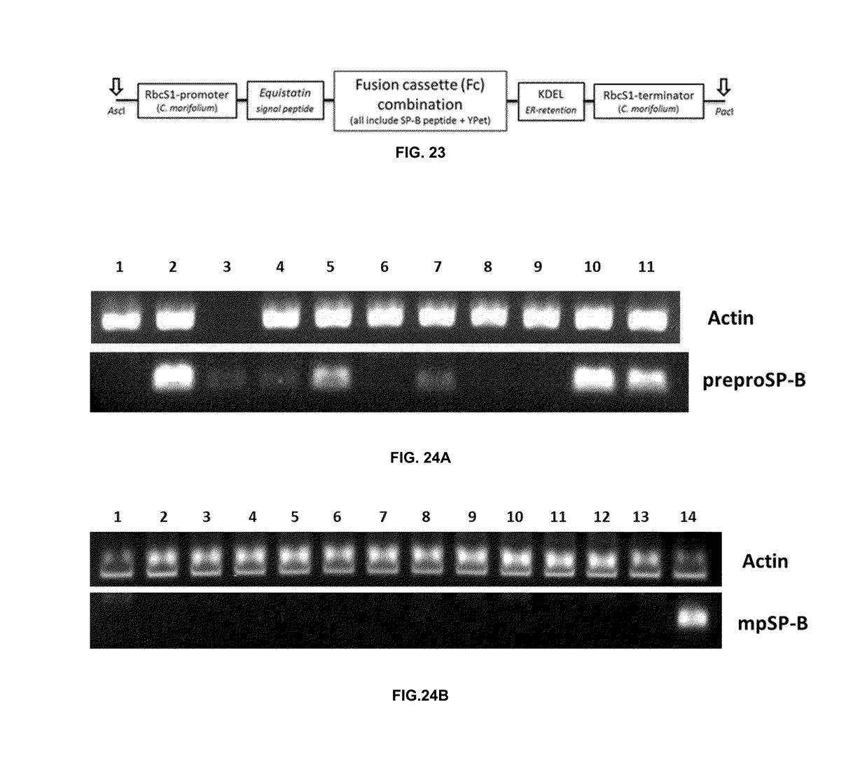

The His-Tag®-mpSP-B fusion was amplified including the 6× His-Tag®, but excluding the native 5′-ATG from pET14B-mpSP-B using the primers: NtSP27-FRW (5′-CCAAGCTTCATCATCATCATCATCACAG-3′) (SEQ ID NO: 29) and NtSP27-REV (5′-TCTAGATCACATGGAGCACCGGAGGACGA-3′) (SEQ ID NO: 30) generating HindIII and XbaI sites at the 5′- and 3′ ends respectively. PCR amplification using Expand® enzyme mix and pET14B-mpSP-B ligation mix as template, included predenaturation at 95° C. for 5 min followed by 30 cycles of amplification (95° C. denaturation, 30 sec; 55° C. annealing, 30 sec; 72° C. polymerization, 30 sec). PCR products were extracted and cloned in a pGEM®-T Easy Vector (Promega Corporation, Madison, USA) to yield pGEM-His-NtSP-mpSP-B. Positive colonies were identified by restriction digest with HindIII and XbaI, excised from the agarose gel and cloned into HindIII/XbaI prepared pART27Nt-SP vector to yield pART27-NtSP-His-mpSP-B (see FIG. 22). Selected yield pART27-NtSP-His-mpSP-B clones were sequenced (ABI PRISM® 3100 genetic analyser) using the ready reaction kit with AmpliTaq® DNA polymerase (The Perkin Elmer Corporation, Norwalk, USA) to confirm NtSP-His-mpSP-B sequence.

### 3. Generation of Transgenic Plants

Tobacco plants were maintained on Murashige Skoog (MS) medium [Murashige and Skoog 1962] in a growth room regulated at a temperature of 22° C. and a 16 h photoperiod. Expression cassettes, pART27_preproSP-B_SEKDEL and pART27-NtSP-His-mpSP-B, were electroporated into Agrobacterium tumefaciens strain LBA4404 [Mattanovich et al 1989] to generate recombinant Agrobacterium tumefaciens. Tobacco leaves (from plants not older than three months) were picked, washed, cut and transformed with the recombinant Agrobacterium tumefaciens using a standard leaf disc method [Horsch et al 1985]. Plantlets were regenerated under kanamycin selection on MS medium and primary transgenic tobacco plantlets generated were hardened off and grown in a containment glasshouse under standard glasshouse conditions at 22° C.

### 4. Molecular Analysis and RT-PCR of Transgenic Plant Material

To evaluate transgenic status of tobacco plants, DNA was extracted from leaf material according to Sambrook et al., [1989] and purified using the Wizard® Genomic DNA Purification Kit (Promega Corporation, Madison, USA). PCR, using primers: SP-B_frw (5′-TCGAGTTCCCCATTCCTCTCCC-3′) (SEQ ID NO: 31) and SP-B_rev (5′-AGACCAGCTGGGGCAGCATG-3′) (SEQ ID NO: 32), consisted of a predenaturation cycle at 94° C. for 2 min followed by 35 cycles of amplification (94° C. denaturation, 30 sec; 55° C. annealing, 30 sec; 72° C. polymerization, 30 sec) to amplify a 211 bp fragment.

For RT-PCR analysis, total RNA was isolated using the RNeasy® Plant Mini Kit (Qiagen) according to manufacturer's instructions. RNA was treated with DNAse I using the NucleoSpin® RNA II kit according to manufacturer's protocol (MACHEREY-NAGEL). First strand cDNA synthesis and reverse transcriptase-PCR was carried out using the PrimeScript™ One Step RT-PCR Kit Ver.2 (Takara Bio) with the same primers used for transgenic analysis to isolate a 211 bp fragment from both preproSP-B and mpSP-B transcripts respectively. RT-PCR included an incubation reaction at 50° C. for 30 min and inactivation at 94° C. for 2 min followed by 35 cycles of amplification (94° C. denaturation, 30 sec; 60° C. annealing, 30 sec; 72° C. polymerization, 40 sec). PCR reactions were repeated with β-actin primers as ‘housekeeping’ control and PCR products were visualized in ethidium bromide-stained 1.2% (w/v) agarose gels. All PCR reactions were carried out in a Perkin-Elmer GeneAmp® Thermocycler 9700 (Perkin Elmer Corporation, Wellesley, USA).

### 5. Protein Purification, SDS-PAGE and Western Blot Analysis

Extraction Method 1 (Acetone and LEW Buffer):

One gram plant tissue was washed 4× with 20 ml acetone to remove plant pigments. The tissue samples were allowed to dry after removal of the acetone. Five mL extraction buffer (LEW buffer from Protino Ni-TED kit-MACHEREY-NAGEL)+1% triton and 30 mM−mercaptoethanol, was added and the proteins extracted for 2 hours on ice. The supernatant was collected by centrifugation at 12 000 rpm and the proteins precipitated with 50% ice cold acetone on ice for 2 hours. The precipitated protein was collected by centrifungation at 10 000 rpm and re-suspended in 500 mL extraction buffer supplemented with 100 mM DTT.

Extraction Method 2 (Methanol Chloroform):

Plant tissue was extracted as described in U.S. Pat. No. 6,172,203 [Hager and DePaoli, 2001] with modifications. 2 Grams of plant leaf tissue ground up in liquid nitrogen and extracted with 40 ml of 100% acetone to remove pigments, supernatant removed and acetone allowed to evaporate at 65 degrees C. Dried material was extracted with 10 ml of Chloroform:Methanol (2:1) and supernatant collected. 5 ml of 0.1M NaCl was used to extract the pellet and the supernatants combined. After centrifugation the organic phase was collected and back extracted with 5 ml of NaCl and organic phase collected and precipitated with 6 volumes of acetone.

Extraction Method 3 (Phenol):

Plant tissue was extracted as described in Wang et al., 2006 with or with the addition of 5% Beta Mercapthoethanol to all steps of the extraction.

Chromogenic Detection:

Proteins were separated on a 12.5% SDS-PAGE gel and electroblotted to PVDF membrane. The membrane was blocked with 5% skim milk in TBS-T for overnight followed by primary antibody (1:1000 dilution in 1% BSA in TBS-T) for 4 hours. SF-B was detected with secondary antibody diluted (1:10000) in TBS-T and the ECL kit from Amersham.

Chemiluminescent Detection:

Proteins were separated on a 8-16% Tris-Hepes SDS-PAGE gel and electroblotted to PVDF membrane. The membrane was blocked with Pierce Superblock for chemiluminescent detection for 16 hours followed by primary antibody (Abnova H00006439-B01P at a 1:20000 dilution in Superblock or Hycult HP9049 diluted 1:500 in superblock) for 1 hour. SF-B was detected with secondary antibody (Hycult HP1202 diluted 1:5000 in superblock) and Pierce Supersignal west Pico substrate according to the instructions of the manufacturer. Abnova H00006439-P01 and protein extracted from commercially obtained Curosurf® surfactant was used as positive controls.

### 6. Peptide Sequencing

Crude protein was excised from the SDS-PAGE gel and digested with trypsin. Mass spectrometry experiments were performed on a Nano-LC EASY-Column system connected to a LTQ Orbitrap Velos mass spectrometer (Thermo Fisher Scientific, Bremen, Germany).

### 7. Results

### 7.1 Transgenic and RT-PCR Analysis

The applicants were able to demonstrate that the majority of transgenic plant lines, under kanamycin selection, confirmed presence of transgene (results not shown). Semi-quantitative RT-PCR analysis revealed transcriptional activity for cDNA's encoding both preproSP-B and mpSP-B protein isoforms respectively.

Seven preproSP-B lines and one mpSP-B line demonstrated activity on mRNA level (see FIGS. 24A and B).

### 7.2. Western Blots

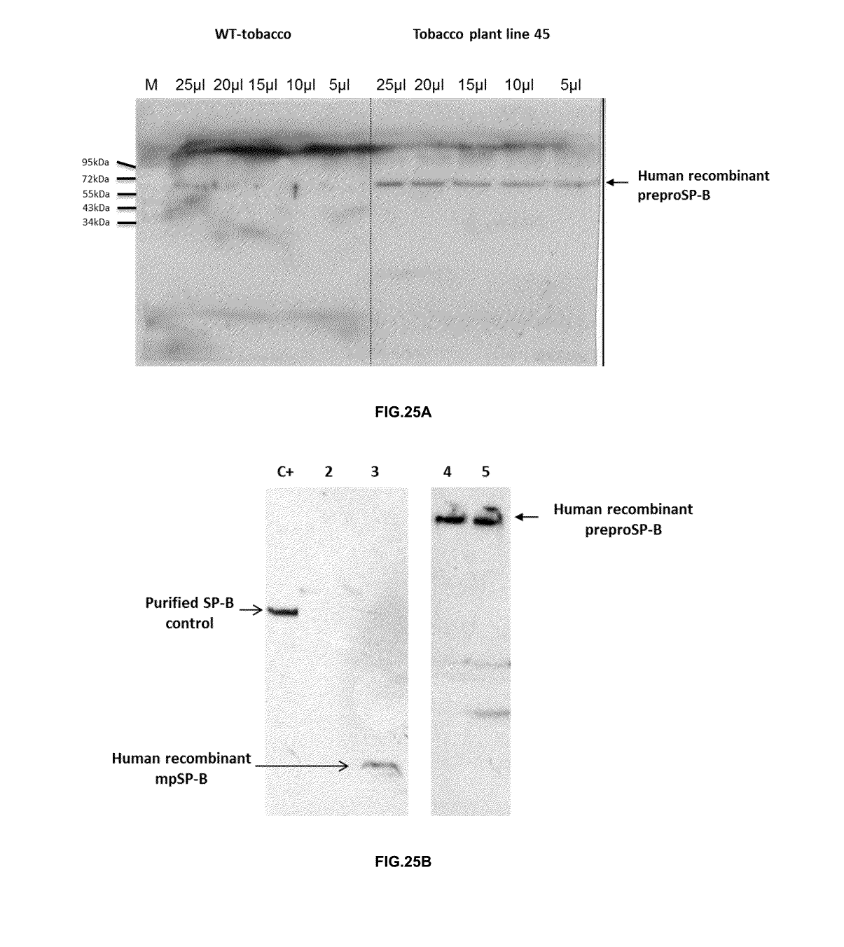

As illustrated in FIG. 25A, the western blot showed expression of human recombinant preproSP-B for the extraction method using acetone and LEW buffer.

Using the Phenol extraction method (see FIG. 25B), preproSP-B protein expression was demonstrated in preproSP-B plant line leaf tissue and mpSP-B protein expression in mpSP-B plant line leaf tissue respectively. The size of the preproSP-B protein bands detected, compared to purified SP-B control, indicates the possible formation of disulfide or other complexes.

Methanol:cloroform (2:1) extraction was also used to successfully extract mpSP-B protein from plant line leaf tissue (results not shown).

### 7.3 Peptide Sequencing

Sequence analysis of crude protein extracts, excised from the SDS-PAGE gel and digested with trypsin revealed presence of a peptide YSVILLDTLLGR. Analysis of this peptide using UniProt database (www.uniprot.gov/blast) scored 100% identity to surfactant protein-B (results not shown).

### Example 2

### 1. Materials and Methods

### 1.1 Fusion Cassette (Fc) Design Strategy, Synthesis and Cloning in Plant Transformation Vector

All protein fusion cassettes comprised single or double copies of the human mature (8 kDa) SP-B peptide and the YPet fluorescent protein which is a yellow fluorescent protein (YFP) modified for Forster resonance energy transfer (FRET) applications [Nguyen and Daugherty 2005]. Some fusion partners were chosen for their ability to be easily extracted via affinity chromatography while others were chosen for their solubility. Still others were chosen to enhance overall expression levels of the cassettes for easy detection in plants. The aim of the various fusion combinations was to find the optimal combination for easy detection and easy extraction in a water soluble form. All fusions were separated by linker peptide(s) containing a protease recognition site that would allow splicing of the fusion partners from the SP-B peptide. Recognition sites included in each cassette was either from tobacco etch virus (TEV), or chymosin, or both. Synthesis and sub-cloning of all fusion cassette sequence combinations were conducted by DNA2.0 (www.dna20.com). Fusion cassettes were synthesized and cloned into the NcoI/BglII sites of ImpactVector 1.3 (Plant Research International, Wageningen; (www.pri.wur.nl/UK/products/ImpactVector/). Within ImpactVector 1.3 each fusion expression cassette contains an N-terminal signal peptide from sea anemone (Actinia equina) equistatin, driven by the RbcS1 promoter of Chrysanthemum morifolium and a C-terminal KDEL sequence for endoplasmic reticulum (ER) retention and a terminator of rbcS1 gene from C. morifolium (see FIG. 22). Subsequently, each fusion expression cassette was excised using AscI and PacI restriction enzymes and re-cloned into the plant binary vector pCambia 1300 (CAMBIA, Canberra, Australia), of which the multiple cloning site was modified by DNA2.0 to accommodate AscI and PacI restriction and ligation.

1.2 Fusion Cassette (Fc) Designs from 5′ to 3′

Six fusion cassettes were designed with various selected fusion partners based on the desired characteristics as set out above and then tested for expression in transgenic tobacco plant lines to determine whether the fusion cassettes would indeed result in successful expression of the fusion proteins of the expected size in transgenic tobacco plants.

### Combination 1 (Designated as Fc_3):

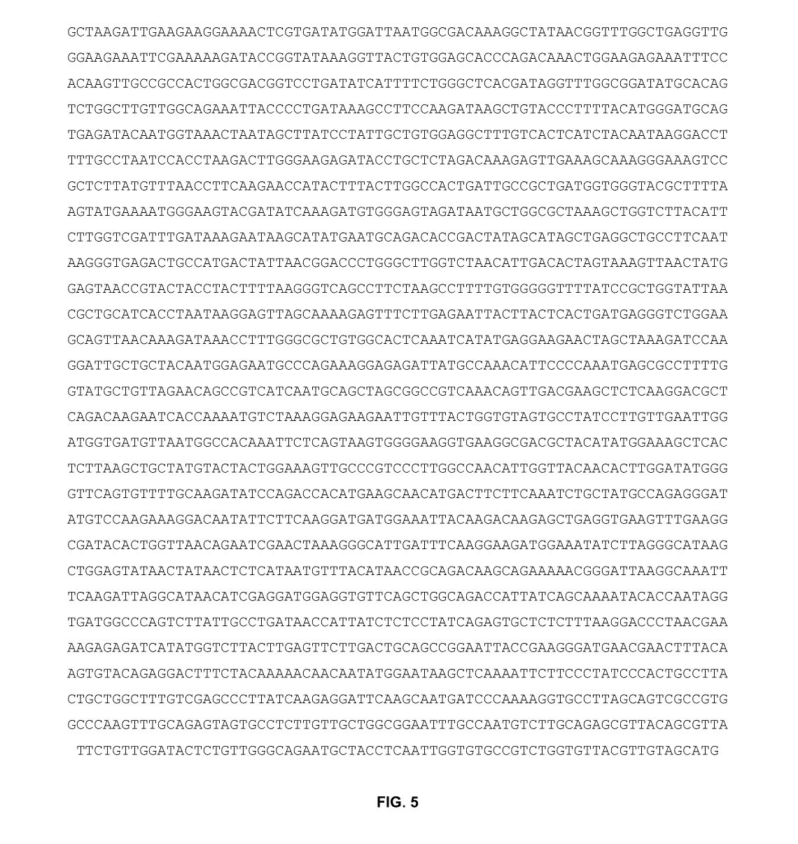

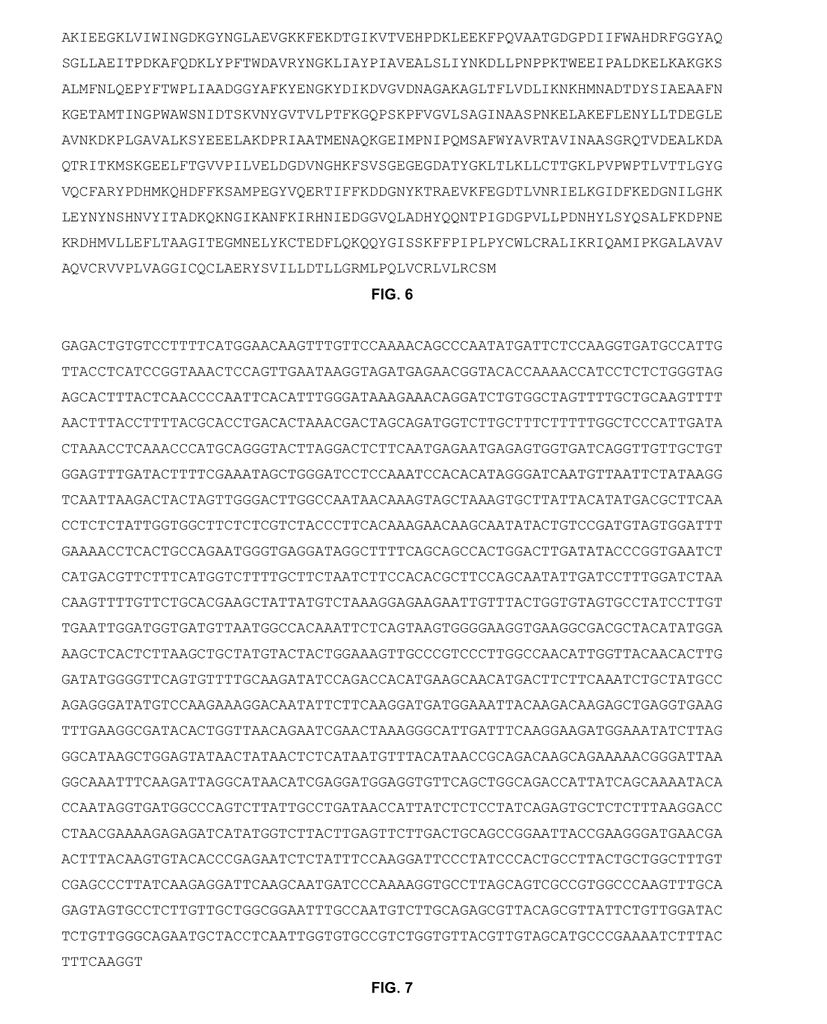

Maltose-binding protein (MBP)_Ypet_chymosin cleaving site_SP-B (FIGS. 5 and 6). Fc_3 has an expected size of 80.73 kDa and contains a MBP fusion partner for enhanced solubility, correct folding and purification of target peptide.

### Combination 2 (Designated as Fc_7):

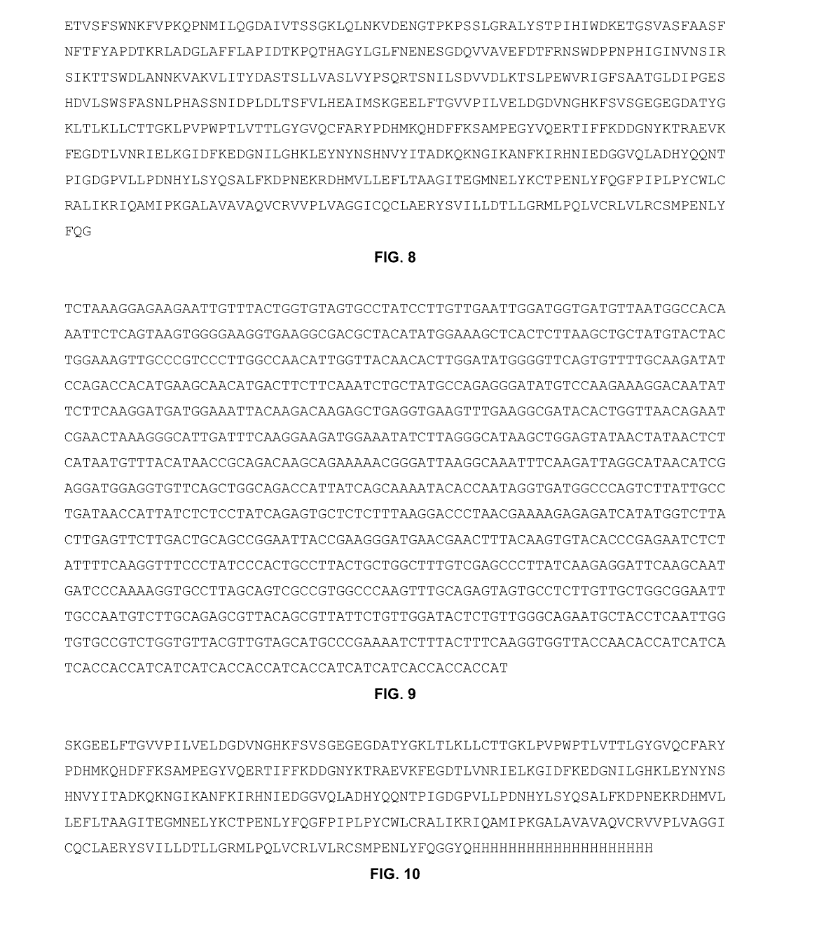

Lectin_YPet_TEV cleaving site_SP-B_TEV cleaving site (FIGS. 7 and 8). Fc_7 has an expected size of 67.39 kDa and contains a soybean (Glycine max) lectin protein fusion partner to serve as an affinity tag for rapid purification.

### Combination 3 (Designated as Fc_9):

YPet_TEV cleaving site_SP-B_TEV cleaving site_poly-Histidine-tag (FIGS. 9 and 10). Fc_9 has an expected size of 40.6 kDa and contains a polyhistidine-tag of twenty (20) histidine repeats for rapid and relative inexpensive purification of the target peptide via metal affinity chromatography.

### Combination 4 (Designated as Fc_10):

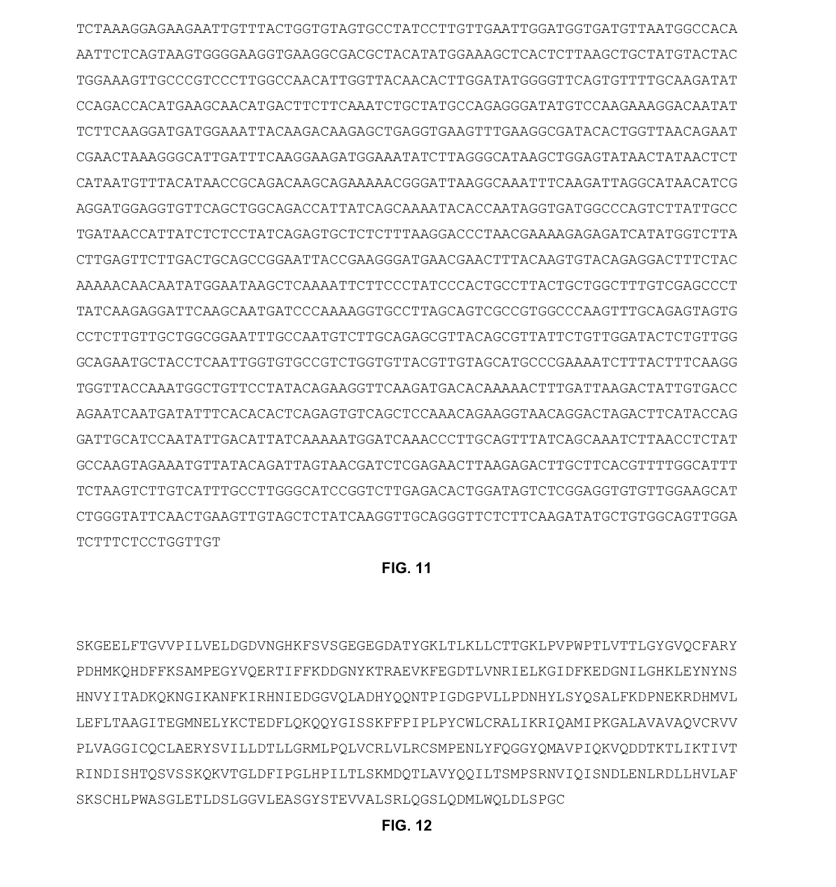

YPet_chymosin cleaving site_SP-B_TEV cleaving site_Leptin (FIGS. 11 and 12). Fc 10 has an expected size of 55.89 kDa and contains a human leptin protein which is a product of the obese gene for enhanced target peptide stability and expression level.

### Combination 5 (Designated as Fc_12):

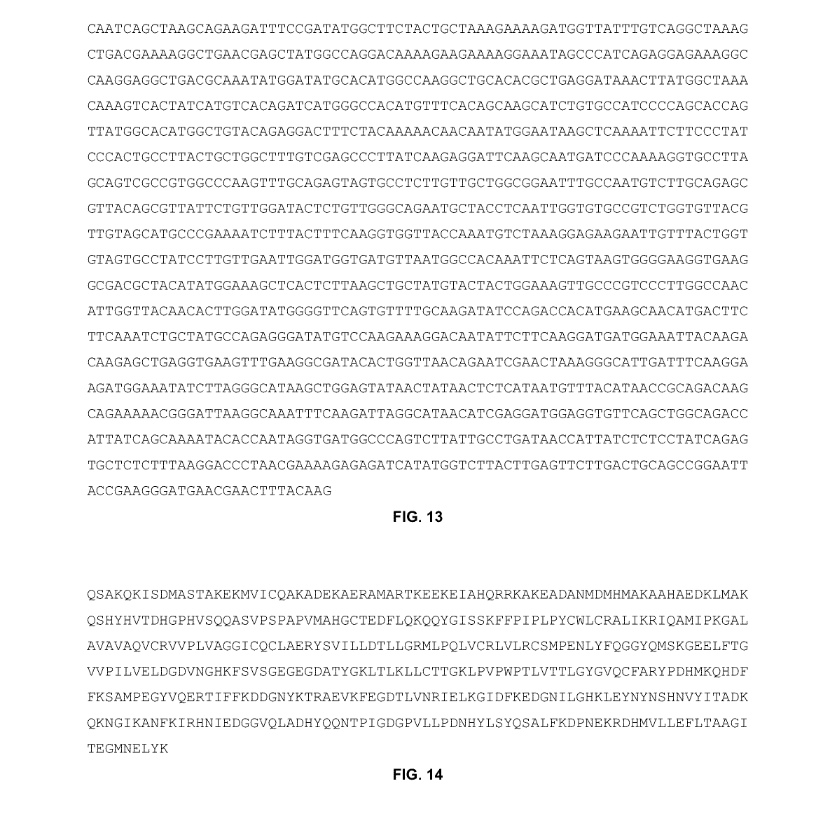

Late Embryogenesis Abundant (LEA) protein_chymosin cleaving site_SP-B_TEV cleaving site_YPet (FIGS. 13 and 14). Fc_12 has an expected size of 50.91 kDa and contains an Arabidopsis thaliana LEA protein fusion partner for improved stability of the expressed protein and enhanced expression levels in response to water and temperature stress.

### Combination 6 (Designated as Fc_13):

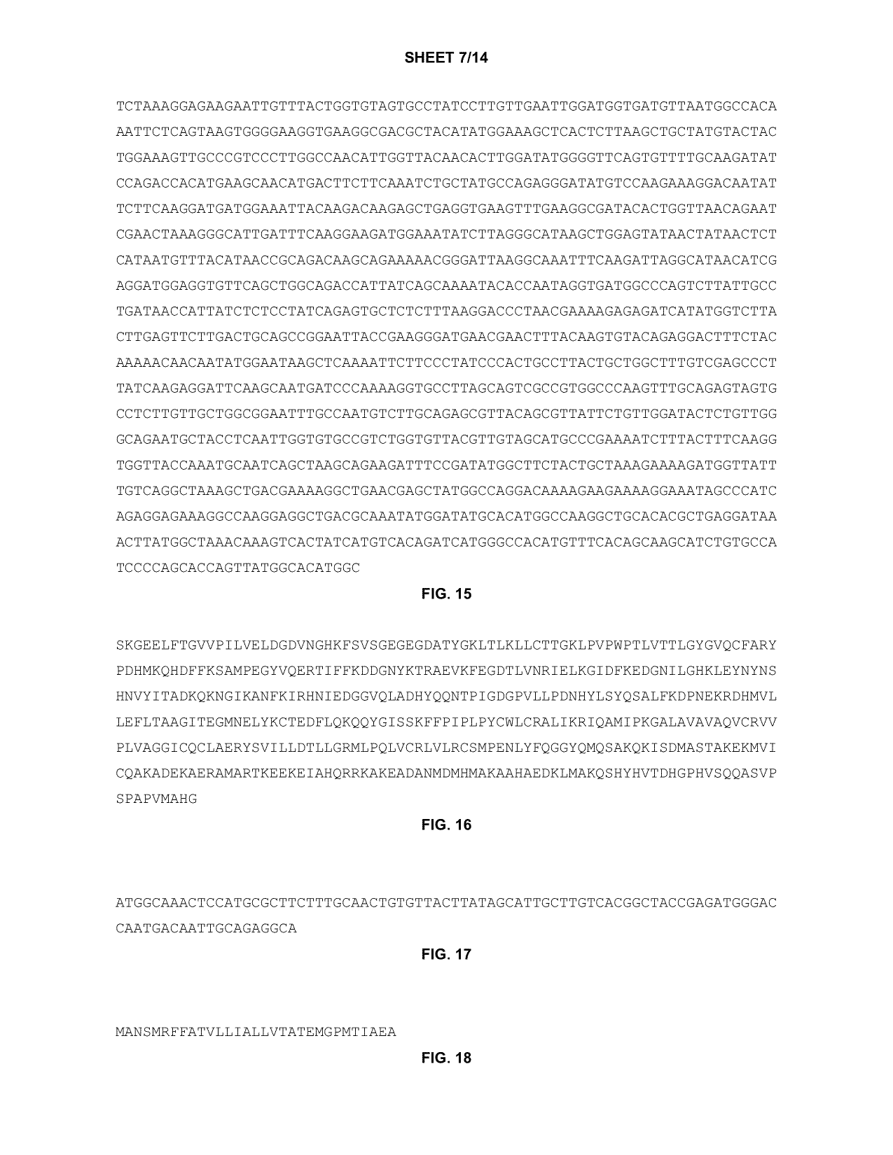

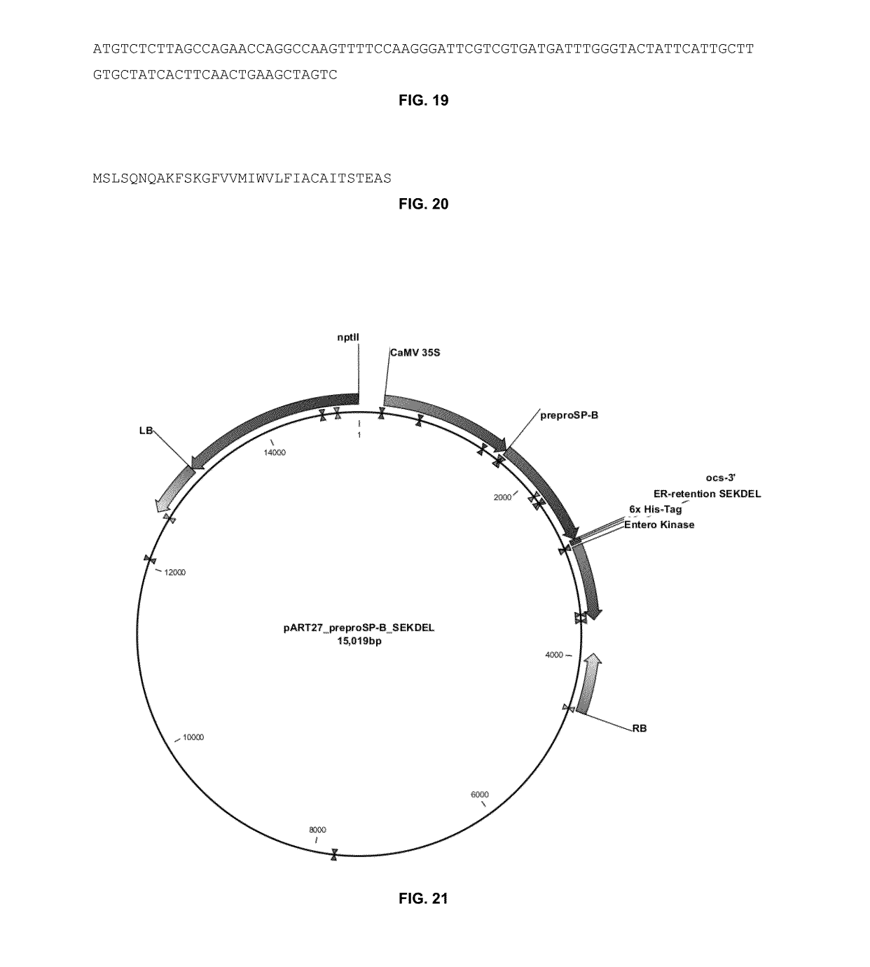

YPet_chymosin cleaving site_SP-B_TEV cleaving site_Late Embryogenesis Abundant (LEA) protein (FIGS. 15 and 16). Fc_13 has the same fusion partners as Fc_12 but with a C-terminal LEA protein.

### 1.3 Generation of Transgenic Plants and Genomic PCR

Tobacco plants (Nicotiana tabacum var Samsun) were maintained on MS medium [Murashige and Skoog 1962] in a temperature (22° C.) regulated growth room at a 16 h photoperiod. The pCambia expression vectors comprising fusion cassettes Fc_3, 7, 9, 10, 12 and 13 were introduced into Agrobacterium tumefaciens strain LBA4404 via electroporation [Mattanovich et al 1989]. Tobacco leaves (from young plants) were transformed using a standard leaf disc method [Horsch et al 1985]. Tobacco plantlets were regenerated under kanamycin selection (150 μg/mL) on MS medium and primary transgenic plantlets were hardened off and grown in a containment glasshouse under standard glasshouse conditions at 22° C. To evaluate transgenic status of tobacco plants, genomic DNA was extracted from leaf material using the GeneJET Genomic DNA Purification Kit (Thermo Fisher Scientific Inc, USA). PCR, using primers: YPet_frw (5′-CTCAGTAAGTGGGGAAGGTGAAGGC-3′) (SEQ ID NO: 33) and YPet_rev (5′-TGCCAGCTGAACACCTCCATCCTCG-3′) (SEQ ID NO: 34), consisted of a predenaturation cycle at 94° C. for 2 min followed by 35 cycles of amplification (94° C. denaturation, 30 sec; 55° C. annealing, 30 sec; 72° C. polymerization, 30 sec) to amplify a 457 bp fragment using GoTaq® DNA Polymerase (Promega Corporation, Madison, USA). All PCR reactions were carried out in a Perkin-Elmer GeneAmp® Thermocycler 9700 (Perkin Elmer Corporation, Wellesley, USA) and PCR products were visualized in ethidium bromide-stained 1.2% (w/v) agarose gels.

### 1.4 Protein Extraction and Western Blot Analysis

Proteins were extracted from the plant tissue with a buffer containing 1×phosphate buffered saline (PBS, Sigma-Aldrich, 79383) with 7M Urea, 2M Thiourea, 5% CHAPS and 5% Beta Mercaptho Ethanol. 500 mg of plant leaf material that was ground up in liquid Nitrogen was extracted with 1 ml of extraction buffer. The extract was vortexed for 1 min and left on the bench for 10 minutes where after it was vortexed again and centrifuged for 10 min at 13000×g. The supernatant was used directly to load into the gel for western blot analysis. SDS PAGE was performed with the Life technologies Bolt™ system (Life Technologies) using Bis-Tris MOPS buffer and 4-12% gradient gel. Transfer and western blot was performed with the Life technologies iBlot® system according to the instructions of the manufacturer. Chromogenic detection was done using the iBlot® chromogenic western detection kit (Life Technologies, 1B7410-01). All transgenic plant lines from each fusion protein cassette were screened with an antibody that specifically recognises GFP mutants (Thermo Fischer Scientific, PA1-28521). To serve as a positive control, fluorescent protein (YPet) was extracted from transgenic plants transformed with pCambia-ImpactVector 1.1 of which RbcS1 promoter and equistatin signal peptide was replaced with double enhancer CaMV 35S promoter only expressing YPet.

### 1.5 Isolation and Purification of SF-B Peptide

His-tag purified sample in solution from construct Fc_9 (GFP-His-tag fusion cassette) was prepared for N-terminal peptide sequencing. Protein extract was digested with trypsin. Smaller peptide sequences were generated and compared to protein sequences in international databases (UniPROT and SwissPROT).

### 2. Results

### 2.1 Western Blot Analysis

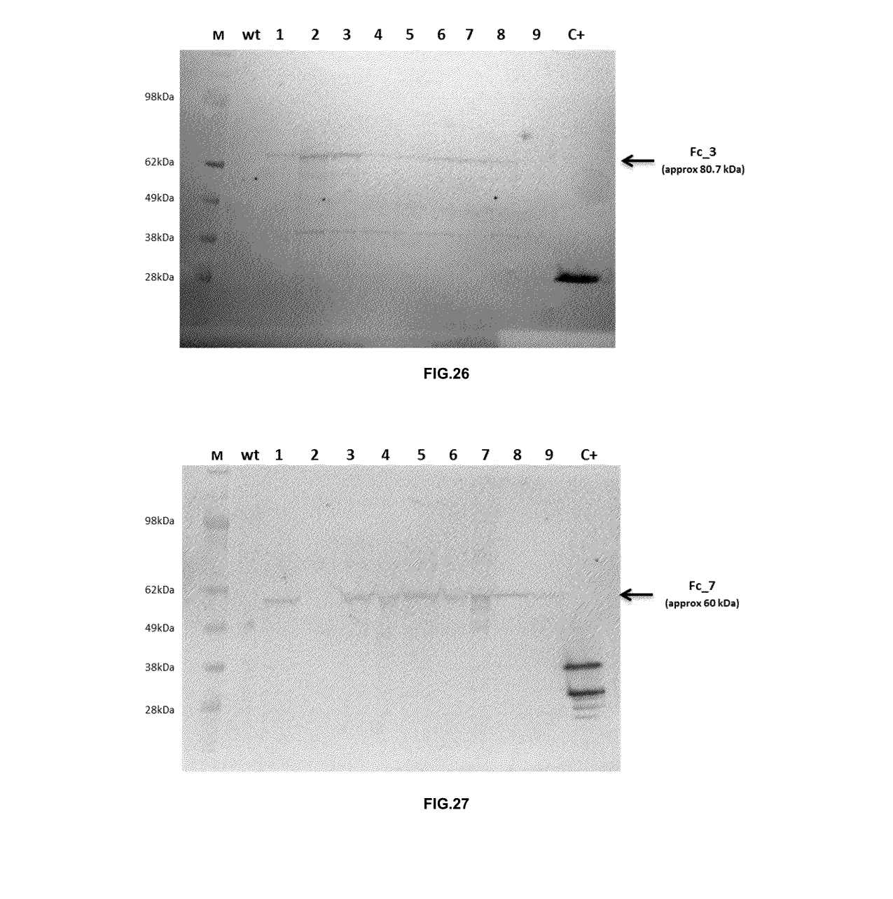

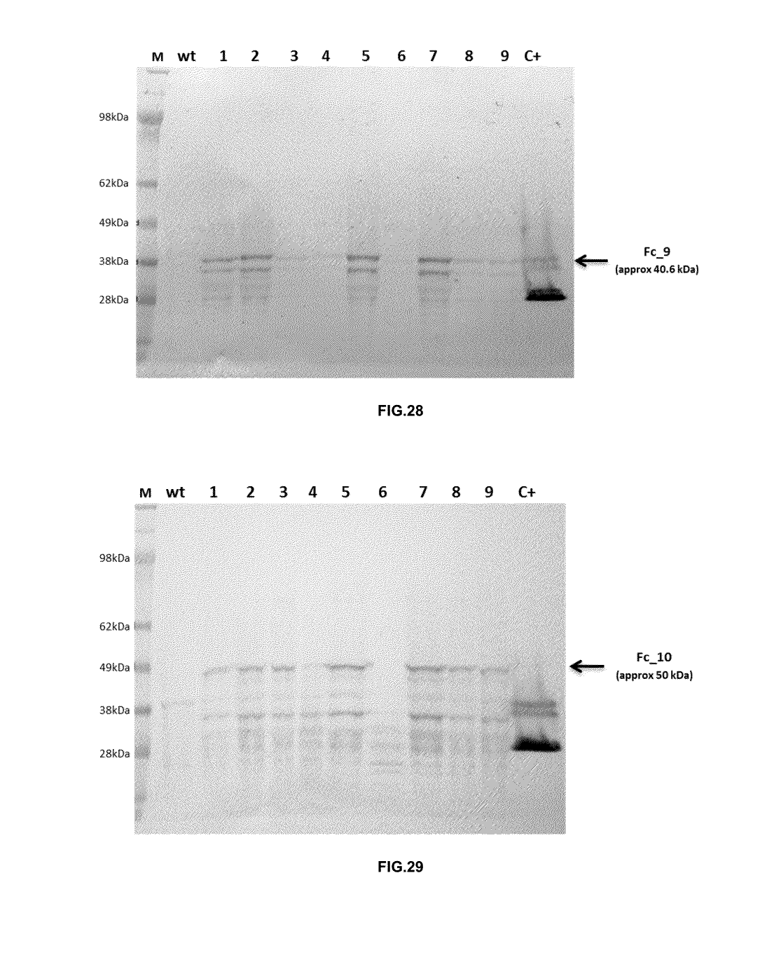

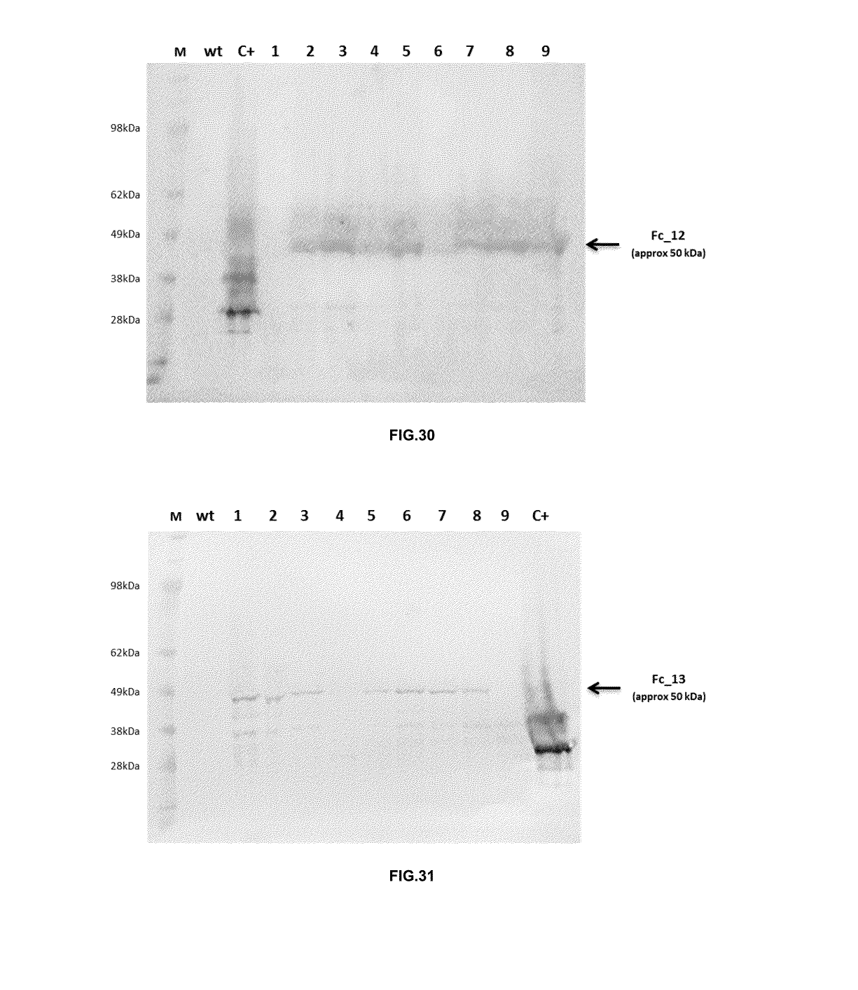

Western blot analysis showed GFP antibody detection in most transgenic plant lines for all fusion cassettes at the expected fusion protein size when compared to the SeeBlue® Plus2 Pre-Stained protein standard (Life Technologies, LC5925) as set out in FIGS. 25 to 30. GFP antibody detects positive control, fluorescent protein (YPet), at approximately 28 kDa. Detection of the fusion cassettes using GFP antibody was specific, however, for some cassettes the sizes revealed to be slightly smaller than expected when compared to the protein standard marker. This could be due to the folding of the protein fusion complex within the endoplasmic reticulum. Nine (9) independent plant lines were analysed for each Fc, except for Fc_3 for which sixteen (16) plant lines were analysed.

### 2.2 Isolation and Purification of SF-B Peptide

Sequence analysis of purified His-tag protein extract in solution from construct Fc_9 (GFP-His-tag fusion cassette) and digested with trypsin, revealed the presence of three peptides: IQAMIPK, VVPLVAGGICQCLAER and YSVILLDTLLGR. Analysis of these peptides using UniProt and SwissPROT databases showed that all peptides scored 100% identity to mature human SP-B.

### REFERENCES

Almlen A, Walther F J, Waring A J, Robertson B, Johansson J, Curstedt T (2010) Synthetic surfactant based on analogues of SP-B and SP-C is superior to single-peptide surfactants in ventilated premature rabbits. Neonatology 98(1): 91-99
Blanco O, Lugones Y, Fernandez O, Faure R (2012) An update on clinical surfactant preparations and respiratory disease. Biotecnologia Aplicada 29: 53-59
Clark H, Clark L S (2005) The genetics of neonatal respiratory disease. Seminars in Fetal and Neonatal Medicine 10(3): 271-282
Cockshutt A M, Possmayer F (1992) Metabolism of surfactant lipids and proteins in the developing lung. In: Robertson B, van Golde L M G, Batenburg J J (eds) Pulmonary surfactant: From Molecular Biology to Clinical Practice. Elsevier Science Publishers, Amsterdam, pp 339-378
Creuwels L A J M, Van Golde L M G, Haagsman H P (1997) The pulmonary surfactant system: Biochemical and clinical aspects. Lung 175: 1-39
Drugmand J-C, Schneider Y-J, Agathos S N (2012) Insect cells as factories for biomanufacturing. Biotechnology Advances 30(5): 1140-1157
Gleave A P (1992) A versatile binary vector system with a T-DNA organisational structure conducive to efficient integration of cloned DNA into the plant genome. Plant Molecular Biology 20: 1203-1207
Goerke J (1998) Pulmonary surfactant: functions and molecular composition. Biochem Biophys Acta 1408: 79-89
Guttentag S, Foster C D (2011) Update in surfactant therapy. NeoReviews 12: e625-e633
Sambrook J, Fritsch E F, Maniatis T (1989) Molecular Cloning: A laboratory manual. 2nd ed. Cold Spring Harbor Laboratory Press, Cold Spring Harbor.
Halliday H L (2008) Surfactants: past present and future. Journal of Perinatology 28: S47-S56
Wilson D F, Notter R H (2011) The future of exogenous surfactant therapy. Respiratory Care 56(9): 1369-1386
Ma C C-H, Ma S (2012) The role of surfactant in respiratory distress syndrome. The Open Respiratory Medicine Journal 6: 44-53
Pryhuber G S (1998) Regulation and function of pulmonary surfactant protein B. Molecular Genetics and Metabolism 64: 217-228
Jobe A H (1993) Pulmonary surfactant therapy. New England Journal of Medicine 328:861-868.
Mallory G B (2001) Surfactant proteins: Role in lung physiology and disease in early life. Paediatric Respiratory Reviews 2 (2): 151-158
Haataja R, Hallman M (2002) Surfactant proteins as genetic determinants of multifactorial pulmonary diseases. Annals of Medicine 34(5): 324-333
Whitsett J A, Weaver T E (2002) Hydrophobic surfactant proteins in lung function and disease. New England Journal of Medicine 347(26): 2141-2148
Griese M (1999) Pulmonary surfactant in health and human lung diseases: State of the art. European Respiratory Journal 13(6): 1455-1476
Weaver T E, Conkright J J (2001) Functions of surfactant proteins B and C. Annual Review of Physiology 63: 555-578
Stevens T P, Sinkin R A (2007) Surfactant replacement therapy. Chest 131(5): 1577-1582
Finer N N (2004) Surfactant use for neonatal lung injury: Beyond respiratory distress syndrome. Paediatric Respiratory Reviews 5(SUPPL. A): S289-S297
Lusuardi M, Capelli A, Carli S, Tacconi M T, Salmona M, Donner C F (1992) Role of surfactant in chronic obstructive pulmonary disease: Therapeutic implications. Respiration 59(SUPPL. 1): 28-32
Kim J Y, Kim Y-G, Lee G M (2012) CHO cells in biotechnology for production of recombinant proteins: Current state and further potential. Applied Microbiology and Biotechnology 93(3): 917-930
Mattanovich D, Branduardi P, Dato L, Gasser B, Sauer M, Porro D (2012) Recombinant protein production in yeasts. Methods in Molecular Biology 824: 329-358
Murashige T, Skoog F (1962) A revised medium for rapid growth and bio assays with tobacco tissue cultures. Plant Physiology 15:473-497
Huang C-J, Lin H, Yang X (2012) Industrial production of recombinant therapeutics in Escherichia coli and its recent advancements. Journal of Industrial Microbiology and Biotechnology 39(3): 383-399
Engle W A, Stark A R, Adamkin D H, Batton D G, Bell E F, Bhutani V K, Denson S E, Martin G I, Watterberg K L (2008) Surfactant-replacement therapy for respiratory distress in the preterm and term neonate. Pediatrics 121(2): 419-432
Seehase M, Collins J J P, Kuypers E, Jellema R K, Ophelders D R M G, Ospina O L, Perez-Gil J, Bianco F, Garzia R, Razzetti R, Kramer B W (2012) New surfactant with SP-B and SP-C analogs gives survival benefit after inactivation in preterm lambs. PLoS ONE 7(10): Article no. 47631
Mingarro I, Lukovic D, Vilar M, Pérez-Gil J (2008) Synthetic pulmonary surfactant preparations: New developments and future trends. Current Medicinal Chemistry 15(4): 393-403
Mattanovich D, Ruker F, da Camara Machado A, Laimer M, Regner F, Steinkeliner H, Himmler G, Katinger H (1989) Efficient transformation of Agrobacterium spp. by eletroporation. Nucleic Acids Research 17(16):6747
Horsch R, Fry J, Hofmann N, Eichhlotz D, Rogers S, Fraylet R (1985) A simple and general method for transferring genes into plants. Science 227(4691): 1229-1231
Parra E, Moleiro L H, Lopez-Montero I, Cruz A, Monroy F, Perez-Gil J (2011) A combined action of pulmonary surfactant proteins SP-B and SP-C modulates permeability and dynamics of phospholipid membranes. Biochemical Journal 438(3): 555-564
Lukovic D, Plasencia I, Taberner F J, Salgado J, Calvete J J, Perez-Gil J, Mingarro I (2006) Production and characterisation of recombinant forms of human pulmonary surfactant protein C (SP-C): Structure and surface activity. Biochimica et Biophysica Acta-Biomembranes 1758(4): 509-518
Jordan B K, Donn S M (2013) Lucinactant for the prevention of respiratory distress syndrome in premature infants. Expert Review of Clinical Pharmacology 6(2): 115-121
Berlec A, Strukelj B (2013) Current state and recent advances in biopharmaceutical production in Escherichia coli, yeasts and mammalian cells. Journal of Industrial Microbiology and Biotechnology 40(3-4): 257-274
Yurdakok M (2004) Inherited disorders of neonatal lung diseases. Turkish Journal of Pediatrics 46(2): 105-114
Zuo Y Y, Veldhuizen R A W, Neumann A W, Petersen N O, Possmayer F (2008) Current perspectives in pulmonary surfactant-Inhibition, enhancement and evaluation. Biochimica et Biophysica Acta-Biomembranes 1778(10): 1947-1977
Hager, A. A., and DePaoli, T. (2001). Method for extracting and purifying pulmonary surfactant
Wang, W., Vignani, R., Scali, M., and Cresti, M. (2006). A universal and rapid protocol for protein extraction from recalcitrant plant tissues for proteomic analysis. Electrophoresis 27, 2782-2786

1. A method of producing a protein in a plant cell, wherein the protein is selected from the group consisting of pulmonary surfactant protein-B (SP-B) pre-protein or a functional fragment or analog thereof, and SP-B mature peptide or a functional fragment or analog thereof, the method comprising the steps of:
(i) providing a nucleic acid sequence comprising a polynucleotide sequence encoding the protein;
(ii) introducing the nucleic acid sequence into an expression vector adapted to express a polypeptide in a plant cell;
(iii) introducing the expression vector of step (ii) into a plant cell;
(iv) expressing the protein in the plant cell of step (iii); and
(v) harvesting the protein from the plant cell.

2. The method according to claim 1, wherein the SP-B pre-proprotein, or SP-B mature peptide, or fragment thereof is encoded by a human gene or is produced from a protein encoded by a human gene.

3. The method according to claim 1, wherein the polynucleotide sequence encoding the SP-B pre-proprotein, or SP-B mature peptide is:
a) 100% identical to any one of SEQ I.D. NOs 2 or 4;
b) a variant sequence at least 80% identical to any one of SEQ I.D. NOs 2 or 4; or
c) a sequence which hybridises under stringent conditions to the reverse complement of any one of SEQ I.D. NOs 2 or 4,
wherein the variant sequence or sequence which hybridises under stringent conditions to the reverse complement encodes a polypeptide capable of lowering surface tension of a lipid bilayer membrane on interaction with phospholipids.

4. The method according to claim 3, wherein the number of hydrophobic amino acid residues encoded by the variant sequence or sequence which hybridises under stringent conditions to the reverse complement of any one of SEQ I.D. NOs 2 or 4 is the same as, or greater than, the number of hydrophobic amino acid residues encoded by any one of SEQ I.D. NOs 2 or 4.

5. The method according to claim 3, wherein the polynucleotide sequence has 100% sequence identity to any one of the polynucleotide sequences set out as SEQ I.D. NOs 2 or 4.

6. The method according to claim 1, wherein the encoded SP-B pre-proprotein, or SP-B mature peptide sequence has 100% sequence identity to any one of SEQ I.D. NOs 1 or 3, or is a variant sequence at least 90% identical to any one of SEQ I.D. NOs 1 or 3, wherein the variant sequence is capable of lowering surface tension of a lipid bilayer membrane on interaction with phospholipids.

7. The method according to claim 6, wherein the number of hydrophobic amino acid residues in the variant sequence is the same as, or greater than, the number of hydrophobic amino acid residues in any one of SEQ I.D. NOs 1 or 3.

8. The method according to claim 6, wherein the SP-B pre-proprotein, or SP-B mature peptide sequence has 100% sequence identity to any one of the polypeptide sequences set out as SEQ ID NOs: 1 or 3.

9. The method according to claim 1, wherein the nucleic acid sequence further comprises any one or more of the following elements:
(i) a polynucleotide sequence encoding a protease cleavage site, selected from an enterokinase, a chymosin and a Tobacco Etch Virus (TEV) protease cleavage site;
(ii) a polynucleotide sequence encoding a tag selected from a polyhistidine, leptin, late embryogenesis abundant protein (LEA), lectin, maltose binding protein (MBP), and a glutathione S-transferase (GST) tag; and
(iii) a polynucleotide sequence encoding a marker protein for detection, selected from YPet and GFP.

10. The method according to claim 11, wherein the polynucleotide sequence encoding the protein is contained in a fusion cassette comprising one or more elements selected from the group consisting of: MBP; YPet; chymosin; lectin; TEV; polyhistidine; leptin; and LEA.

11. The method according to claim 1, wherein the nucleic acid sequence further comprises any one or more of the following additional elements:
(i) a plant promoter selected from chrysanthemum RbcS1, 35S, CaMV35S, and a corresponding terminator sequence, wherein both the plant promoter and the terminator sequence are operably linked to the polynucleotide sequence the protein;
(ii) a polynucleotide sequence encoding a signal peptide selected from the group consisting of the signal peptide region of equistatin and of Nicotiana tabacum thionin (NtSP);
(iii) a polynucleotide sequence encoding an endoplasmic reticulum (ER)-trafficking peptide; an oil body-trafficking peptide; a protein storage vacuole-trafficking peptide; a plastid trafficking peptide; and
(iv) a polynucleotide sequence comprising a psbA regulatory 5′-UTR and 3′ UTR region for targeting an encoded transcript to a plastid.

12. The method according to claim 11, wherein the fusion cassette consists of any one of the following combinations in the order set out:
(i) MBP-YPet-chymosin-SP-B;
(ii) lectin-YPet-TEV-SP-B-TEV;
(iii) YPet-TEV-SP-B-TEV-polyhistidine;
(iv) YPet-chymosin-SP-B-TEV-leptin;
(v) LEA-chymosin-SP-B-TEV-YPet; and
(vi) YPet-chymosin-SP-B-TEV-LEA.

13. The method according to claim 1,
wherein the expression vector of step (ii) further comprises a polynucleotide sequence encoding a suppressor protein adapted to inhibit post-transcriptional gene silencing in a plant cell, or
wherein step (iii) further includes introducing into the plant cell a second plant vector comprising a polynucleotide sequence encoding a suppressor protein adapted to inhibit post-transcriptional gene silencing in the plant cell,
the suppressor protein being selected from the NSs protein of the tomato spotted wilt virus or the p19 of tomato bushy stunt virus.

14. The method according to claim 1, wherein the nucleic acid sequence further comprises a polynucleotide sequence encoding an endoplasmic reticulum (ER) trafficking peptide selected from SEKDEL or KDEL and in step (iv) the expressed pulmonary surfactant protein-B pre-proprotein is retained in the ER of the plant cell.

15. The method according to claim 1, wherein the plant expression vector of step (ii) is an Agrobacterium tumefaciens vector.

16. The method according to claim 1, wherein the plant expression vector of step (ii) is an Agrobacterium tumefaciens vector and step (iii) of the method comprises stable transformation of a plant cell by the introduction of the expression vector of step (ii) into the genetic material of the plant cell by means of Agrobacterium transformation or agroinfiltration.

17. The method according to claim 1, wherein the plant cell is
one of a plurality of plant cells in suspension culture,
one of plant cells in tissue culture,
one of the plant cells in a leaf of a plant or a transgenic plant or any part thereof.

18. A polypeptide fusion cassette comprising the protein produced by the method of claim 11.

19. An expression vector for expression of a protein in a plant comprising a nucleic acid sequence encoding a protein selected from the group consisting of pulmonary surfactant protein-B (SP-B) pre-protein or a functional fragment or analog thereof, and SP-B mature peptide or a functional fragment or analog thereof.

20. (canceled)

21. A plant cell comprising the protein produced according to the method of claim 1.


SEQUENCE LISTING (30 sequences)

SEQ ID NO: 1 - protein, 381 aa, Homo sapiens
MAESHLLQWLLLLLPTLCGPGTAAWTTSSLACAQGPEFWCQSLEQALQCRALGHCLQEVWGHVGADDLCQECEDIVHILNKMAKEAIFQDTMRKFLEQECNVLPLKLLMPQCNQVLDDYFPLVIDYFQNQTDSNGICMHLGLCKSRQPEPEQEPGMSDPLPKPLRDPLPDPLLDKLVLPVLPGALQARPGPHTQDLSEQQFPIPLPYCWLCRALIKRIQAMIPKGALAVAVAQVCRVVPLVAGGICQCLAERYSVILLDTLLGRMLPQLVCRLVLRCSMDDSAGPRSPTGEWLPRDSECHLCMSVTTQAGNSSEQAIPQAMLQACVGSWLDREKCKQFVEQHTPQLLTLVPRGWDAHTTCQALGVCGTMSSPLQCIHSPDL

SEQ ID NO: 2 - DNA, 1146 bp, Homo sapiens
atggctgagtcacacctgctgcagtggctgctgctgctgctgcccacgctctgtggcccaggcactgctgcctggaccacctcatccttggcctgtgcccagggccctgagttctggtgccaaagcctggagcaagcattgcagtgcagagccctagggcattgcctacaggaagtctggggacatgtgggagccgatgacctatgccaagagtgtgaggacatcgtccacatccttaacaagatggccaaggaggccattttccaggacacgatgaggaagttcctggagcaggagtgcaacgtcctccccttgaagctgctcatgccccagtgcaaccaagtgcttgacgactacttccccctggtcatcgactacttccagaaccagactgactcaaacggcatctgtatgcacctgggcctgtgcaaatcccggcagccagagccagagcaggagccagggatgtcagaccccctgcccaaacctctgcgggaccctctgccagaccctctgctggacaagctcgtcctccctgtgctgcccggggccctccaggcgaggcctgggcctcacacacaggatctctccgagcagcaattccccattcctctcccctattgctggctctgcagggctctgatcaagcggatccaagccatgattcccaagggtgcgctagctgtggcagtggcccaggtgtgccgcgtggtacctctggtggcgggcggcatctgccagtgcctggctgagcgctactccgtcatcctgctcgacacgctgctgggccgcatgctgccccagctggtctgccgcctcgtcctccggtgctccatggatgacagcgctggcccaaggtcgccgacaggagaatggctgccgcgagactctgagtgccacctctgcatgtccgtgaccacccaggccgggaacagcagcgagcaggccataccacaggcaatgctccaggcctgtgttggctcctggctggacagggaaaagtgcaagcaatttgtggagcagcacacgccccagctgctgaccctggtgcccaggggctgggatgcccacaccacctgccaggccctcggggtgtgtgggaccatgtccagccctctccagtgtatccacagccccgacctttga

SEQ ID NO: 3 - protein, 79 aa, Homo sapiens
FPIPLPYCWLCRALIKRIQAMIPKGALAVAVAQVCRVVPLVAGGICQCLAERYSVILLDTLLGRMLPQLVCRLVLRCSM

SEQ ID NO: 4 - DNA, 237 bp, Homo sapiens
ttccccattcctctcccctattgctggctctgcagggctctgatcaagcggatccaagccatgattcccaagggtgcgctagctgtggcagtggcccaggtgtgccgcgtggtacctctggtggcgggcggcatctgccagtgcctggctgagcgctactccgtcatcctgctcgacacgctgctgggccgcatgctgccccagctggtctgccgcctcgtcctccggtgctccatg

SEQ ID NO: 5 - DNA, 2115 bp, Artificial Sequence
  cDNA sequence encoding the fusion cassette consisting of MBP - YPet - chymosin - SP-B
gctaagattgaagaaggaaaactcgtgatatggattaatggcgacaaaggctataacggtttggctgaggttgggaagaaattcgaaaaagataccggtataaaggttactgtggagcacccagacaaactggaagagaaatttccacaagttgccgccactggcgacggtcctgatatcattttctgggctcacgataggtttggcggatatgcacagtctggcttgttggcagaaattacccctgataaagccttccaagataagctgtacccttttacatgggatgcagtgagatacaatggtaaactaatagcttatcctattgctgtggaggctttgtcactcatctacaataaggaccttttgcctaatccacctaagacttgggaagagatacctgctctagacaaagagttgaaagcaaagggaaagtccgctcttatgtttaaccttcaagaaccatactttacttggccactgattgccgctgatggtgggtacgcttttaagtatgaaaatgggaagtacgatatcaaagatgtgggagtagataatgctggcgctaaagctggtcttacattcttggtcgatttgataaagaataagcatatgaatgcagacaccgactatagcatagctgaggctgccttcaataagggtgagactgccatgactattaacggaccctgggcttggtctaacattgacactagtaaagttaactatggagtaaccgtactacctacttttaagggtcagccttctaagccttttgtgggggttttatccgctggtattaacgctgcatcacctaataaggagttagcaaaagagtttcttgagaattacttactcactgatgagggtctggaagcagttaacaaagataaacctttgggcgctgtggcactcaaatcatatgaggaagaactagctaaagatccaaggattgctgctacaatggagaatgcccagaaaggagagattatgccaaacattccccaaatgagcgccttttggtatgctgttagaacagccgtcatcaatgcagctagcggccgtcaaacagttgacgaagctctcaaggacgctcagacaagaatcaccaaaatgtctaaaggagaagaattgtttactggtgtagtgcctatccttgttgaattggatggtgatgttaatggccacaaattctcagtaagtggggaaggtgaaggcgacgctacatatggaaagctcactcttaagctgctatgtactactggaaagttgcccgtcccttggccaacattggttacaacacttggatatggggttcagtgttttgcaagatatccagaccacatgaagcaacatgacttcttcaaatctgctatgccagagggatatgtccaagaaaggacaatattcttcaaggatgatggaaattacaagacaagagctgaggtgaagtttgaaggcgatacactggttaacagaatcgaactaaagggcattgatttcaaggaagatggaaatatcttagggcataagctggagtataactataactctcataatgtttacataaccgcagacaagcagaaaaacgggattaaggcaaatttcaagattaggcataacatcgaggatggaggtgttcagctggcagaccattatcagcaaaatacaccaataggtgatggcccagtcttattgcctgataaccattatctctcctatcagagtgctctctttaaggaccctaacgaaaagagagatcatatggtcttacttgagttcttgactgcagccggaattaccgaagggatgaacgaactttacaagtgtacagaggactttctacaaaaacaacaatatggaataagctcaaaattcttccctatcccactgccttactgctggctttgtcgagcccttatcaagaggattcaagcaatgatcccaaaaggtgccttagcagtcgccgtggcccaagtttgcagagtagtgcctcttgttgctggcggaatttgccaatgtcttgcagagcgttacagcgttattctgttggatactctgttgggcagaatgctacctcaattggtgtgccgtctggtgttacgttgtagcatg

SEQ ID NO: 6 - protein, 705 aa, Artificial Sequence
  polypeptide sequence of the the fusion cassette consisting of MBP - YPet - chymosin - SP-B
AKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITKMSKGEELFTGVVPILVELDGDVNGHKFSVSGEGEGDATYGKLTLKLLCTTGKLPVPWPTLVTTLGYGVQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALFKDPNEKRDHMVLLEFLTAAGITEGMNELYKCTEDFLQKQQYGISSKFFPIPLPYCWLCRALIKRIQAMIPKGALAVAVAQVCRVVPLVAGGICQCLAERYSVILLDTLLGRMLPQLVCRLVLRCSM

SEQ ID NO: 7 - DNA, 1761 bp, Artificial Sequence
  cDNA sequence encoding the fusion cassette consisting of Lectin - YPet - TEV - SP-B - TEV
gagactgtgtccttttcatggaacaagtttgttccaaaacagcccaatatgattctccaaggtgatgccattgttacctcatccggtaaactccagttgaataaggtagatgagaacggtacaccaaaaccatcctctctgggtagagcactttactcaaccccaattcacatttgggataaagaaacaggatctgtggctagttttgctgcaagttttaactttaccttttacgcacctgacactaaacgactagcagatggtcttgctttctttttggctcccattgatactaaacctcaaacccatgcagggtacttaggactcttcaatgagaatgagagtggtgatcaggttgttgctgtggagtttgatacttttcgaaatagctgggatcctccaaatccacacatagggatcaatgttaattctataaggtcaattaagactactagttgggacttggccaataacaaagtagctaaagtgcttattacatatgacgcttcaacctctctattggtggcttctctcgtctacccttcacaaagaacaagcaatatactgtccgatgtagtggatttgaaaacctcactgccagaatgggtgaggataggcttttcagcagccactggacttgatatacccggtgaatctcatgacgttctttcatggtcttttgcttctaatcttccacacgcttccagcaatattgatcctttggatctaacaagttttgttctgcacgaagctattatgtctaaaggagaagaattgtttactggtgtagtgcctatccttgttgaattggatggtgatgttaatggccacaaattctcagtaagtggggaaggtgaaggcgacgctacatatggaaagctcactcttaagctgctatgtactactggaaagttgcccgtcccttggccaacattggttacaacacttggatatggggttcagtgttttgcaagatatccagaccacatgaagcaacatgacttcttcaaatctgctatgccagagggatatgtccaagaaaggacaatattcttcaaggatgatggaaattacaagacaagagctgaggtgaagtttgaaggcgatacactggttaacagaatcgaactaaagggcattgatttcaaggaagatggaaatatcttagggcataagctggagtataactataactctcataatgtttacataaccgcagacaagcagaaaaacgggattaaggcaaatttcaagattaggcataacatcgaggatggaggtgttcagctggcagaccattatcagcaaaatacaccaataggtgatggcccagtcttattgcctgataaccattatctctcctatcagagtgctctctttaaggaccctaacgaaaagagagatcatatggtcttacttgagttcttgactgcagccggaattaccgaagggatgaacgaactttacaagtgtacacccgagaatctctatttccaaggattccctatcccactgccttactgctggctttgtcgagcccttatcaagaggattcaagcaatgatcccaaaaggtgccttagcagtcgccgtggcccaagtttgcagagtagtgcctcttgttgctggcggaatttgccaatgtcttgcagagcgttacagcgttattctgttggatactctgttgggcagaatgctacctcaattggtgtgccgtctggtgttacgttgtagcatgcccgaaaatctttactttcaaggt

SEQ ID NO: 8 - protein, 587 aa, Artificial Sequence
  polypeptide sequence of the the fusion cassette consisting of Lectin - YPet - TEV - SP-B - TEV
ETVSFSWNKFVPKQPNMILQGDAIVTSSGKLQLNKVDENGTPKPSSLGRALYSTPIHIWDKETGSVASFAASFNFTFYAPDTKRLADGLAFFLAPIDTKPQTHAGYLGLFNENESGDQVVAVEFDTFRNSWDPPNPHIGINVNSIRSIKTTSWDLANNKVAKVLITYDASTSLLVASLVYPSQRTSNILSDVVDLKTSLPEWVRIGFSAATGLDIPGESHDVLSWSFASNLPHASSNIDPLDLTSFVLHEAIMSKGEELFTGVVPILVELDGDVNGHKFSVSGEGEGDATYGKLTLKLLCTTGKLPVPWPTLVTTLGYGVQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALFKDPNEKRDHMVLLEFLTAAGITEGMNELYKCTPENLYFQGFPIPLPYCWLCRALIKRIQAMIPKGALAVAVAQVCRVVPLVAGGICQCLAERYSVILLDTLLGRMLPQLVCRLVLRCSMPENLYFQG

SEQ ID NO: 9 - DNA, 1071 bp, Artificial Sequence
  cDNA sequence encoding the fusion cassette consisting of YPet - TEV - SP-B - TEV - polyhistidine
tctaaaggagaagaattgtttactggtgtagtgcctatccttgttgaattggatggtgatgttaatggccacaaattctcagtaagtggggaaggtgaaggcgacgctacatatggaaagctcactcttaagctgctatgtactactggaaagttgcccgtcccttggccaacattggttacaacacttggatatggggttcagtgttttgcaagatatccagaccacatgaagcaacatgacttcttcaaatctgctatgccagagggatatgtccaagaaaggacaatattcttcaaggatgatggaaattacaagacaagagctgaggtgaagtttgaaggcgatacactggttaacagaatcgaactaaagggcattgatttcaaggaagatggaaatatcttagggcataagctggagtataactataactctcataatgtttacataaccgcagacaagcagaaaaacgggattaaggcaaatttcaagattaggcataacatcgaggatggaggtgttcagctggcagaccattatcagcaaaatacaccaataggtgatggcccagtcttattgcctgataaccattatctctcctatcagagtgctctctttaaggaccctaacgaaaagagagatcatatggtcttacttgagttcttgactgcagccggaattaccgaagggatgaacgaactttacaagtgtacacccgagaatctctattttcaaggtttccctatcccactgccttactgctggctttgtcgagcccttatcaagaggattcaagcaatgatcccaaaaggtgccttagcagtcgccgtggcccaagtttgcagagtagtgcctcttgttgctggcggaatttgccaatgtcttgcagagcgttacagcgttattctgttggatactctgttgggcagaatgctacctcaattggtgtgccgtctggtgttacgttgtagcatgcccgaaaatctttactttcaaggtggttaccaacaccatcatcatcaccaccatcatcatcaccaccatcaccatcatcatcaccaccaccat

SEQ ID NO: 10 - protein, 357 aa, Artificial Sequence
  polypeptide sequence of the the fusion cassette consisting of YPet - TEV - SP-B - TEV - polyhistidine
SKGEELFTGVVPILVELDGDVNGHKFSVSGEGEGDATYGKLTLKLLCTTGKLPVPWPTLVTTLGYGVQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALFKDPNEKRDHMVLLEFLTAAGITEGMNELYKCTPENLYFQGFPIPLPYCWLCRALIKRIQAMIPKGALAVAVAQVCRVVPLVAGGICQCLAERYSVILLDTLLGRMLPQLVCRLVLRCSMPENLYFQGGYQHHHHHHHHHHHHHHHHHHHH

SEQ ID NO: 11 - DNA, 1476 bp, Artificial Sequence
  cDNA sequence encoding the fusion cassette consisting of YPet - chymosin - SP-B - TEV - Leptin
tctaaaggagaagaattgtttactggtgtagtgcctatccttgttgaattggatggtgatgttaatggccacaaattctcagtaagtggggaaggtgaaggcgacgctacatatggaaagctcactcttaagctgctatgtactactggaaagttgcccgtcccttggccaacattggttacaacacttggatatggggttcagtgttttgcaagatatccagaccacatgaagcaacatgacttcttcaaatctgctatgccagagggatatgtccaagaaaggacaatattcttcaaggatgatggaaattacaagacaagagctgaggtgaagtttgaaggcgatacactggttaacagaatcgaactaaagggcattgatttcaaggaagatggaaatatcttagggcataagctggagtataactataactctcataatgtttacataaccgcagacaagcagaaaaacgggattaaggcaaatttcaagattaggcataacatcgaggatggaggtgttcagctggcagaccattatcagcaaaatacaccaataggtgatggcccagtcttattgcctgataaccattatctctcctatcagagtgctctctttaaggaccctaacgaaaagagagatcatatggtcttacttgagttcttgactgcagccggaattaccgaagggatgaacgaactttacaagtgtacagaggactttctacaaaaacaacaatatggaataagctcaaaattcttccctatcccactgccttactgctggctttgtcgagcccttatcaagaggattcaagcaatgatcccaaaaggtgccttagcagtcgccgtggcccaagtttgcagagtagtgcctcttgttgctggcggaatttgccaatgtcttgcagagcgttacagcgttattctgttggatactctgttgggcagaatgctacctcaattggtgtgccgtctggtgttacgttgtagcatgcccgaaaatctttactttcaaggtggttaccaaatggctgttcctatacagaaggttcaagatgacacaaaaactttgattaagactattgtgaccagaatcaatgatatttcacacactcagagtgtcagctccaaacagaaggtaacaggactagacttcataccaggattgcatccaatattgacattatcaaaaatggatcaaacccttgcagtttatcagcaaatcttaacctctatgccaagtagaaatgttatacagattagtaacgatctcgagaacttaagagacttgcttcacgttttggcattttctaagtcttgtcatttgccttgggcatccggtcttgagacactggatagtctcggaggtgtgttggaagcatctgggtattcaactgaagttgtagctctatcaaggttgcagggttctcttcaagatatgctgtggcagttggatctttctcctggttgt

SEQ ID NO: 12 - protein, 492 aa, Artificial Sequence
  polypeptide sequence of the the fusion cassette consisting of YPet - chymosin - SP-B - TEV - Leptin
SKGEELFTGVVPILVELDGDVNGHKFSVSGEGEGDATYGKLTLKLLCTTGKLPVPWPTLVTTLGYGVQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALFKDPNEKRDHMVLLEFLTAAGITEGMNELYKCTEDFLQKQQYGISSKFFPIPLPYCWLCRALIKRIQAMIPKGALAVAVAQVCRVVPLVAGGICQCLAERYSVILLDTLLGRMLPQLVCRLVLRCSMPENLYFQGGYQMAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 13 - DNA, 1341 bp, Artificial Sequence
  cDNA sequence encoding the fusion cassette consisting of LEA - chymosin - SP-B - TEV - Ypet
caatcagctaagcagaagatttccgatatggcttctactgctaaagaaaagatggttatttgtcaggctaaagctgacgaaaaggctgaacgagctatggccaggacaaaagaagaaaaggaaatagcccatcagaggagaaaggccaaggaggctgacgcaaatatggatatgcacatggccaaggctgcacacgctgaggataaacttatggctaaacaaagtcactatcatgtcacagatcatgggccacatgtttcacagcaagcatctgtgccatccccagcaccagttatggcacatggctgtacagaggactttctacaaaaacaacaatatggaataagctcaaaattcttccctatcccactgccttactgctggctttgtcgagcccttatcaagaggattcaagcaatgatcccaaaaggtgccttagcagtcgccgtggcccaagtttgcagagtagtgcctcttgttgctggcggaatttgccaatgtcttgcagagcgttacagcgttattctgttggatactctgttgggcagaatgctacctcaattggtgtgccgtctggtgttacgttgtagcatgcccgaaaatctttactttcaaggtggttaccaaatgtctaaaggagaagaattgtttactggtgtagtgcctatccttgttgaattggatggtgatgttaatggccacaaattctcagtaagtggggaaggtgaaggcgacgctacatatggaaagctcactcttaagctgctatgtactactggaaagttgcccgtcccttggccaacattggttacaacacttggatatggggttcagtgttttgcaagatatccagaccacatgaagcaacatgacttcttcaaatctgctatgccagagggatatgtccaagaaaggacaatattcttcaaggatgatggaaattacaagacaagagctgaggtgaagtttgaaggcgatacactggttaacagaatcgaactaaagggcattgatttcaaggaagatggaaatatcttagggcataagctggagtataactataactctcataatgtttacataaccgcagacaagcagaaaaacgggattaaggcaaatttcaagattaggcataacatcgaggatggaggtgttcagctggcagaccattatcagcaaaatacaccaataggtgatggcccagtcttattgcctgataaccattatctctcctatcagagtgctctctttaaggaccctaacgaaaagagagatcatatggtcttacttgagttcttgactgcagccggaattaccgaagggatgaacgaactttacaag

SEQ ID NO: 14 - protein, 447 aa, Artificial Sequence
  polypeptide sequence of the the fusion cassette consisting of LEA - chymosin - SP-B - TEV - Ypet
QSAKQKISDMASTAKEKMVICQAKADEKAERAMARTKEEKEIAHQRRKAKEADANMDMHMAKAAHAEDKLMAKQSHYHVTDHGPHVSQQASVPSPAPVMAHGCTEDFLQKQQYGISSKFFPIPLPYCWLCRALIKRIQAMIPKGALAVAVAQVCRVVPLVAGGICQCLAERYSVILLDTLLGRMLPQLVCRLVLRCSMPENLYFQGGYQMSKGEELFTGVVPILVELDGDVNGHKFSVSGEGEGDATYGKLTLKLLCTTGKLPVPWPTLVTTLGYGVQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALFKDPNEKRDHMVLLEFLTAAGITEGMNELYK

SEQ ID NO: 15 - DNA, 1341 bp, Artificial Sequence
  cDNA sequence encoding the fusion cassette consisting of YPet - chymosin - SP-B - TEV - LEA
tctaaaggagaagaattgtttactggtgtagtgcctatccttgttgaattggatggtgatgttaatggccacaaattctcagtaagtggggaaggtgaaggcgacgctacatatggaaagctcactcttaagctgctatgtactactggaaagttgcccgtcccttggccaacattggttacaacacttggatatggggttcagtgttttgcaagatatccagaccacatgaagcaacatgacttcttcaaatctgctatgccagagggatatgtccaagaaaggacaatattcttcaaggatgatggaaattacaagacaagagctgaggtgaagtttgaaggcgatacactggttaacagaatcgaactaaagggcattgatttcaaggaagatggaaatatcttagggcataagctggagtataactataactctcataatgtttacataaccgcagacaagcagaaaaacgggattaaggcaaatttcaagattaggcataacatcgaggatggaggtgttcagctggcagaccattatcagcaaaatacaccaataggtgatggcccagtcttattgcctgataaccattatctctcctatcagagtgctctctttaaggaccctaacgaaaagagagatcatatggtcttacttgagttcttgactgcagccggaattaccgaagggatgaacgaactttacaagtgtacagaggactttctacaaaaacaacaatatggaataagctcaaaattcttccctatcccactgccttactgctggctttgtcgagcccttatcaagaggattcaagcaatgatcccaaaaggtgccttagcagtcgccgtggcccaagtttgcagagtagtgcctcttgttgctggcggaatttgccaatgtcttgcagagcgttacagcgttattctgttggatactctgttgggcagaatgctacctcaattggtgtgccgtctggtgttacgttgtagcatgcccgaaaatctttactttcaaggtggttaccaaatgcaatcagctaagcagaagatttccgatatggcttctactgctaaagaaaagatggttatttgtcaggctaaagctgacgaaaaggctgaacgagctatggccaggacaaaagaagaaaaggaaatagcccatcagaggagaaaggccaaggaggctgacgcaaatatggatatgcacatggccaaggctgcacacgctgaggataaacttatggctaaacaaagtcactatcatgtcacagatcatgggccacatgtttcacagcaagcatctgtgccatccccagcaccagttatggcacatggc

SEQ ID NO: 16 - protein, 447 aa, Artificial Sequence
  polypeptide sequence of the the fusion cassette consisting of YPet - chymosin - SP-B - TEV - LEA
SKGEELFTGVVPILVELDGDVNGHKFSVSGEGEGDATYGKLTLKLLCTTGKLPVPWPTLVTTLGYGVQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALFKDPNEKRDHMVLLEFLTAAGITEGMNELYKCTEDFLQKQQYGISSKFFPIPLPYCWLCRALIKRIQAMIPKGALAVAVAQVCRVVPLVAGGICQCLAERYSVILLDTLLGRMLPQLVCRLVLRCSMPENLYFQGGYQMQSAKQKISDMASTAKEKMVICQAKADEKAERAMARTKEEKEIAHQRRKAKEADANMDMHMAKAAHAEDKLMAKQSHYHVTDHGPHVSQQASVPSPAPVMAHG

SEQ ID NO: 17 - DNA, 93 bp, Nicotiana tabacum
atggcaaactccatgcgcttctttgcaactgtgttacttatagcattgcttgtcacggctaccgagatgggaccaatgacaattgcagaggca

SEQ ID NO: 18 - protein, 31 aa, Nicotiana tabacum
MANSMRFFATVLLIALLVTATEMGPMTIAEA

SEQ ID NO: 19 - DNA, 100 bp, Actinia equina
atgtctcttagccagaaccaggccaagttttccaagggattcgtcgtgatgatttgggtactattcattgcttgtgctatcacttcaactgaagctagtc

SEQ ID NO: 20 - protein, 33 aa, Actinia equina
MSLSQNQAKFSKGFVVMIWVLFIACAITSTEAS

SEQ ID NO: 21 - DNA, 21 bp, Artificial Sequence
  preproSP-B_frw primer sequence
atggctgagtcacacctgctg

SEQ ID NO: 22 - DNA, 23 bp, Artificial Sequence
  preproSP-B_rev primer sequence
tcaaaggtcggggctgtggatac

SEQ ID NO: 23 - DNA, 31 bp, Artificial Sequence
  preproSP-B27_frw primer sequence
ccccgaattcatggctgagtcacacctgctg

SEQ ID NO: 24 - DNA, 87 bp, Artificial Sequence
  preproSP-B27_rev primer sequence
ggggaagctttcaaagctcgtccttctcgctgtgatggtggtggtgatgtttgtcatcgtcatcaaggtccgggctgtggatacact

SEQ ID NO: 25 - DNA, 28 bp, Artificial Sequence
  mpSP-B_frw primer sequence
ggctcgagttccccattcctctccccta

SEQ ID NO: 26 - DNA, 29 bp, Artificial Sequence
  mpSP-B_rev primer sequence
ggcagatctcatggagcaccggaggacga

SEQ ID NO: 27 - DNA, 32 bp, Artificial Sequence
  NtSP-FRW primer sequence
ggctcgagatggcaaactccatgcgcttcttt

SEQ ID NO: 28 - DNA, 28 bp, Artificial Sequence
  NtSP-REV primer sequence
ccaagctttgcctctgcaattgtcattg

SEQ ID NO: 29 - DNA, 28 bp, Artificial Sequence
  NtSP27-FRW primer sequence
ccaagcttcatcatcatcatcatcacag

SEQ ID NO: 30 - DNA, 29 bp, Artificial Sequence
  NtSP27-REV
tctagatcacatggagcaccggaggacga